(19) United States
(12) Patent Application Publication
(10) Pub. No.: US 2023/0044475 A1
(43) Pub. Date: Feb. 9, 2023

(54) COMPOSITIONS AND METHODS INVOLVING AMINO ACIDS FOR THE TREATMENT OF FAT INFILTRATIONS IN MUSCLE

(71) Applicant: AXCELLA HEALTH INC., Cambridge, MA (US)

(72) Inventors: Manu Chakravarthy, Newton, MA (US); William Comb, Melrose, MA (US)

(21) Appl. No.: 17/788,481

(22) PCT Filed: Dec. 23, 2020
(86) PCT No.: PCT/US2020/066890

§ 371 Date: Jun. 23, 2022

Related U.S. Application Data
(60) Provisional application No. 62/952,941, filed on Dec. 23, 2019.

(51) Int. Cl. A61K 31/198; A61P 1/16; A61P 21/00; A61P 43/00; A61K 31/4172; A61K 38/05; A61K 38/06; A61K 31/19; A61K 49/06; A23L 33/175

(52) U.S. Cl. CPC A61K 31/198; A61P 1/16; A61P 21/00; A61P 43/00; A61K 31/4172; A61K 38/05; A61K 38/06; A61K 31/19; A61K 49/06; A23L 33/175; A23V 2002/00

(57) ABSTRACT

This disclosure provides methods of using compositions comprising amino acid entities to reduce fat infiltration in muscle, particularly under conditions of muscle dysfunction. The disclosure also provides methods for enhancing muscle function by reducing fat infiltration in the muscle comprising administering an effective amount of the compositions to a subject in need thereof.

### CROSS-REFERENCE TO RELATED APPLICATIONS

This application claims priority under 35 U.S.C. § 119(e) to U.S. Provisional Patent Application Ser. No. 62/952,941, the disclosure of which is hereby incorporated herein by reference in its entirety for all purposes.

### BACKGROUND

There are many diseases and disorders associated with increased infiltration of fat in muscle, which impairs muscle function. Fat infiltration in muscle is associated with cancer, AIDS, renal failure, liver disease, spinal cord injury, congestive heart failure, and cirrhotic sarcopenia. Furthermore, disuse of muscles through immobilization or aging also results in muscle atrophy with increased fat infusion in muscle.

The liver plays a central role in amino acid metabolism and, consequently, patients with liver disease show abnormalities in their plasma amino acid profile (Fischer 1976), specifically, a decrease in branched-chain amino acid (BCAA) levels and a corresponding increase in aromatic amino acid (AAA) levels (Dam 2011).

Ingestion of protein meals (ranging from 20 g to 70 g protein) or just BCAA alone (˜30-35 g) in cirrhotic subjects have been associated with a transient increase (˜30-40% increase over baseline) in plasma ammonia (Campollo 2017; Dam 2011). However, the response of plasma ammonia and key structural (lean tissue mass) and functional (strength, balance) changes to a concurrent administration of a defined mixture of amino acids and a protein meal in subjects with mild to moderate hepatic insufficiency, remained unknown. An earlier study with different amounts of CS-EMM-001 (14.7 g/day and 44.1 g/day for 15 days each) (see US Patent Publication No. 2019/0046487 A1), assessed standard safety chemistry, hematology, ammonia, lean mass, strength and balance measurements in a monitored, domiciled setting in Childs A and B subjects. Daily consumption of CS-EMM-001 at 14.7 or 44.1 g/day for 15 days each was found to be safe and well tolerated, with supraphysiological changes in plasma concentration of the constituent amino acids within CS-EMM-001. Subjects consuming the higher amounts of study product (44.1 g/day) tended to maintain a leaner phenotype with a lowered liver frailty index (LFI) score. However, the effect on fat infiltration into muscle was unknown. Furthermore, the composition of CS-EMM-001 was different from another EMM (endogenous modulators of metabolism, e.g., amino acids) composition that demonstrated reduction of muscle fat infiltration in the context of muscle immobilization (see U.S. patent application Ser. No. 16/446,328, filed Jun. 19, 2019).

The gold standard for treating muscle atrophy conditions is recovery of function. However, direct assessment of muscle functional muscle mass is challenging (Evans et al., 2019, Journal of Cachexia, Sarcopenia and Muscle, 10:14-21). Furthermore, the ability to reduce infiltration of fat in muscle is an important therapeutic target, with few if any pharmacological agents available.

U.S. patent application Ser. No. 16/446,328 discloses a first discovery of the ability of a complex mixture of EMMs to reduce fat infiltration into muscle in the context of muscle immobilization, among other functional effects. Other EMM combinations, such as CS-EMM-001, achieve multifactorial effects for pathologies of liver cirrhosis, including hepatic encephalopathy. Thus, there is a need to identify additional other multifactorial pharmacological methods of reducing fat infiltration in muscle, and to use such agents to reduce fat infiltration in muscle, particularly under conditions of sarcopenia. Furthermore, there is a need to identify markers of functional muscle mass to develop additional metabolic rebalancing compositions for enhancing muscle mass and function (relative to not receiving any therapy), such as for treating muscle-related disease and disorders where fat infiltration in muscle occurs.

### SUMMARY

The invention provides method for reducing fat infiltration in muscle comprising administering to a subject at risk of fat infiltration in muscle a composition comprising an Active Moiety. In one aspect, the composition, e.g., Active Moiety, e.g., Composition 1, comprises:

i) a histidine amino acid entity;

ii) a lysine amino acid entity; and

iii) a threonine amino acid entity.

In some embodiments, the composition does not include a physiologically effective amount of an amino acid entity selected from a leucine amino acid entity, an isoleucine amino acid entity, a valine amino acid entity, a glutamine amino acid entity, an arginine amino acid entity, and a N-acetylcysteine amino acid entity, with the proviso that the composition is not a composition of all essential amino acids (EAAs). In one embodiment, the total wt. % of (i)-(iii) is greater than the total wt. % of any other single amino acid entity in the composition. In one embodiment, the subject suffers from cirrhosis of the liver. Some subjects, e.g., patients with cirrhosis, have elevated levels of serum phenylalanine, which allows omission of this component from a composition for use in a method of the invention. In situations where the subject wherein the subject does not have elevated plasma levels of phenylalanine relative to a normal range of plasma phenylalanine from the Human Metabolomics DataBase (HMDB), the composition (e.g., Active Moiety) further includes a phenylalanine amino acid moiety. In a specific embodiment, the subject suffers from a condition other than cirrhosis of the liver. The composition for use in the method may be a pharmaceutical composition and further comprise a pharmaceutically acceptable excipient or excipients.

The present invention further include a composition (e.g., an Active Moiety) comprising (i) a histidine amino acid entity; (ii) a lysine amino acid entity; and (iii) a threonine amino acid entity; wherein the composition does not include a physiologically effective amount of an amino acid entity selected from a leucine amino acid entity, an isoleucine amino acid entity, a valine amino acid entity, a glutamine amino acid entity, an arginine amino acid entity, and a N-acetylcysteine amino acid entity. In a specific embodiment, wherein the total wt. % of (i)-(iii) is greater than the total wt. % of any other single amino acid entity in the composition. As noted above, where the composition will be administered to a subject who does not have elevated levels of phenylalanine relative to a normal range of plasma phenylalanine from the Human Metabolomics DataBase (HMDB), the composition (Active Moiety) may further include a phenylalanine amino acid entity.

In some embodiments, a composition (e.g., Active Moiety) does not include any of one, two, three, four, five, or six amino acid entities selected from a leucine amino acid entity, an isoleucine amino acid entity, a valine amino acid entity, a glutamine amino acid entity, an arginine amino acid entity, and a N-acetylcysteine amino acid entity. In a further embodiment, such compositions may not include a physiologically effective amount of any of one, two, three, four, five, or six amino acids selected of leucine, isoleucine, valine, glutamine, arginine, and N-acetylcysteine (e.g., one or more of leucine, isoleucine, valine, glutamine, arginine, and N-acetylcysteine if present, is present at less than: 1 wt. %, 0.5 wt. %, 0.1 wt. %, 0.05 wt. %, 0.01 wt. %, 0.001 wt. %, or less, e.g., of the total wt. of the composition (e.g., in dry form). In some embodiments, a composition does not include any of each of leucine, isoleucine, valine, glutamine, arginine, and N-acetylcysteine.

A composition (e.g., Active Moiety) may also be characterized by at least one of methionine (M), tryptophan (W), or cysteine (C) being absent, or if present, is present at less than a physiologically effective amount (e.g., at least one of methionine, tryptophan, or cystein is absent or, if present, is present at less than: 1 wt. %, 0.5 wt. %, 0.1 wt. %, 0.05 wt. %, 0.01 wt. %, 0.001 wt. %, or less, e.g., of the total wt. of the composition (e.g., in dry form). In another aspect, the composition, e.g., Active Moiety, e.g., Composition 2, comprises:

a) a Branched Chain Amino Acid (BCAA) entity chosen from a leucine amino acid entity, an isoleucine amino acid entity, a valine amino acid entity, or a combination of two or three BCAA entities;

b) a Urea Cycle Amino Acid (UCAA) entity chosen from an ornithine amino acid entity, an aspartate amino acid entity, or a combination of two UCAA entities; and

c) an essential amino acid (EAA) entity chosen from a histidine amino acid entity, a lysine amino acid entity, or a threonine amino acid entity or a combination of two or three EAA entities;

wherein at least one amino acid entity (e.g., two, three, four, five, six, seven, or eight amino acid entities) of (a)-(c) is not provided as a peptide of more than 20 amino acid residues in length.

In some embodiments, the ornithine amino acid entity is chosen from L-ornithine, ornithine α-ketoglutarate, ornithine HCl, citrulline, or a combination thereof.

In some embodiments, one, two, or all of phenylalanine, tyrosine, and glutamine are absent from the composition, or if present, are present at less than: 5 wt. %, 1 wt. %, 0.5 wt. %, 0.1 wt. %, 0.05 wt. %, 0.01 wt. %, 0.001 wt. %, or less, e.g., of the total wt. of the composition (in dry form).

In some embodiments, the total wt. % of (a)-(c) or (i)-(iii) (e.g., three, four, five, six, seven, or eight amino acid entities in (a)-(c) or (i)-(iii)) is greater than the total wt. % of other non-amino acid entity protein components (e.g., whey protein), other amino acid entity components, or non-protein components (or two or all of the aforementioned) in the composition on a dry weight basis, e.g., the total wt. % (a)-(c) or (i)-(iii) is at least: 50 wt. %, 75 wt. %, or 90 wt. % of the total wt. of amino acid entities or total components in the composition (in dry form).

In some embodiments, three, four, five, six, seven, or eight amino acid entities in (a)-(c) or (i)-(iii) are in one or both of free amino acid form or salt amino acid form in the composition, e.g., at least: 35 wt. %, 40 wt. %, 42 wt. %, 45 wt. %, 50 wt. %, 75 wt. %, 80 wt. %, 90 wt. %, or more, of the total wt. of the composition (in dry form) is three, four, five, six, seven, or eight amino acid entities in (a)-(c) or (i)-(iii) in one or both of free amino acid form or salt amino acid form in the composition.

In some embodiments, the composition comprises a combination of 19 or fewer, 18 or fewer, 15 or fewer, 12 or fewer, or 10 or fewer amino acid entities. In some embodiments, the combination comprises at least: 42 wt. %, 75 wt. %, or 90 wt. % of the total wt. of amino acid entities or total components in the composition (in dry form).

In some embodiments, one, two, or more (e.g., all) of phenylalanine, tyrosine, or glutamine is absent from the composition, or if present, are present at less than: 10 wt. %, 5 wt. %, 1 wt. %, 0.5 wt. %, 0.1 wt. %, 0.05 wt. %, 0.01 wt. %, 0.001 wt. %, or less, e.g., of the total wt. of the composition (in dry form). In some embodiments, one, two, or more (e.g., all) of phenylalanine, tyrosine, or glutamine, if present, are present in one or both of free amino acid form, or salt amino acid.

In some embodiments, the wt. % of the BCAA entities is at least 37 wt. %, 38 wt. %, 39 wt. %, 40 wt. %, 41 wt. %, 42 wt. %, 43 wt. %, 44 wt. %, 45 wt. %, or more of the total wt. of amino acid entities or total components in the composition (in dry form).

In some embodiments, the wt. % of the UCAA entities is at least 25 wt. %, 26 wt. %, 27 wt. %, 28 wt. %, 29 wt. %, 30 wt. %, 31 wt. %, 32 wt. %, 33 wt. %, 34 wt. %, 35 wt. %, or more of the total wt. of amino acid entities or total components in the composition (in dry form).

In some embodiments, the wt. % of the EAA entities is at least 16 wt. %, 17 wt. %, 18 wt. %, 19 wt. %, 20 wt. %, or more of the total wt. of amino acid entities or total components in the composition (in dry form).

A significant advantage of the invention is the ability to finely regulate the amount and relative ratio of each amino acid in the composition, which is not possible with peptides of more than 20 amino acids in length, including proteins. In some embodiments, the composition does not comprise a peptide of more than 20 amino acid residues in length (e.g., whey protein), or if a peptide of more than 20 amino acid residues in length is present, the peptide is present at less than: 10 weight (wt.) %, 5 wt. %, 1 wt. %, 0.5 wt. %, 0.1 wt. %, 0.05 wt. %, 0.01 wt. %, 0.001 wt. %, or less of the total wt. of amino acid entities or total components of the composition (in dry form).

In some embodiments, the composition has one, two, or three of the following features:

d) the wt. % of the combination of three of the BCAA entities is greater than the wt. % of the UCAA entity or the combination of two of the UCAA entities, e.g., the wt. % of the combination of three of the BCAA entities is at least 5% greater than the wt. % of the UCAA entity or the combination of two of the UCAA entities; e.g., the wt. % of the combination of three of the BCAA entities is at least 10%, 15%, 20%, or more greater than the wt. % of the UCAA entity or the combination of two of the UCAA entities;

e) the wt. % of the combination of three of the BCAA entities is greater than the wt. % of the EAA entity or the combination of two or three of the EAA entities in (c); e.g., the wt. % of the combination of three of the BCAA entities is at least 30% greater than the wt. % of the EAA entity or the combination of two or three of the EAA entities in (c); e.g., the wt. % of the combination of three of the BCAA entities is at least 40%, 50%, or 55%, or more greater than the wt. % of the EAA entity or the combination of two or three of the EAA entities in (c);

f) the wt. % of the combination of the UCAA entity or two of the UCAA entities is greater than the wt. % of the EAA entity or the combination of two or three of the EAA entities in (c); e.g., the wt. % of the UAA entity or the combination of two of the UCAA entities is at least 25% greater than the wt. % of the EAA entity or the combination of two or three of the EAA entities in (c); e.g., the wt. % of the UCAA entity or the combination of two of the UCAA entities is at least 30%, 35%, 40%, or more greater than the wt. % of the EAA entity or the combination of two or three of the EAA entities in (c); or

g) a combination of two or three of (d)-(f).

In some embodiments, a wt. ratio of the BCAA entity or BCAA entities:the UCAA entity or UCAA entities:the EAA entity or EAA entities in (c) is 20+/−15%:15+/−15%:9+/−15%, where the ratios are determined based on an equivalent amount of each amino acid in free form.

In some embodiments, three, four, five, six, seven, or eight amino acid entities in (a)-(c) or (i)-(iii) is selected from Table 1.

In some embodiments, the Active Moiety comprises: a) a leucine amino acid entity chosen from: i) L-leucine or a salt thereof, ii) a dipeptide or salt thereof, or tripeptide or salt thereof, comprising L-leucine, or iii) β-hydroxy-β-methylbutyrate (HMB) or a salt thereof; b) one or both of: i) an ornithine amino acid entity chosen from L-ornithine or a salt thereof or a dipeptide or salt thereof, or tripeptide or salt thereof, comprising L-ornithine; or ii) an aspartate amino acid entity chosen from L-aspartate or a salt thereof or a dipeptide or salt thereof, or tripeptide or salt thereof, comprising L-aspartate; and c) an EAA entity chosen from: i) L-histidine or a salt thereof, ii) a dipeptide or salt thereof, or tripeptide or salt thereof, comprising L-histidine, iii) L-lysine or a salt thereof, iv) a dipeptide or salt thereof, or tripeptide or salt thereof, comprising L-lysine, v) L-threonine or a salt thereof, or vi) a dipeptide or salt thereof, or tripeptide or salt thereof, comprising L-threonine. In some embodiments, the composition further comprises one or both of an isoleucine amino acid entity or a valine amino acid entity, wherein one or both of the isoleucine amino acid entity or the valine amino acid entity is not provided as a peptide of more than 20 amino acid residues in length.

In some embodiments, the isoleucine amino acid entity is L-isoleucine or a salt thereof or a dipeptide or salt thereof, or tripeptide or salt thereof, comprising L-isoleucine. In some embodiments, the valine amino acid entity is L-valine or a salt thereof or a dipeptide or salt thereof, or tripeptide or salt thereof, comprising L-valine.

In some embodiments, a wt. ratio of the leucine amino acid entity:the isoleucine amino acid entity:the valine amino acid entity:the ornithine amino acid entity:the aspartate amino acid entity:the histidine amino acid entity:the threonine amino acid entity:the lysine amino acid entity is 8+/−20%:4+/−20%:8+/−20%:7.5+/−20%:7.5+/−20%:3+/−20%:3+/−20%:3+/−20%, where the ratios are determined based on an equivalent amount of each amino acid in free form.

In some embodiments, the composition comprises: L-leucine or a salt thereof, L-isoleucine or a salt thereof, L-valine or a salt thereof, L-ornithine or a salt thereof, L-aspartate or a salt thereof, L-histidine or a salt thereof, L-threonine or a salt thereof, and L-lysine or a salt thereof (e.g., L-lysine acetate).

According to the invention, any method can be practiced with a composition that is a pharmaceutical composition. Thus, the composition can further comprise a pharmaceutically acceptable excipient, such as an excipient that is suitable for oral administration. In some embodiments, the composition (e.g., the Active Moiety) is formulated with a pharmaceutically acceptable carrier. In some embodiments, the composition (e.g., the Active Moiety) is formulated as a dietary composition. In some embodiments, the dietary composition is chosen from a medical food, a functional food, or a supplement.

Compositions (e.g., Active Moieties) of the invention, which can be used in the methods of the invention, can be characterized by the ability to reduce fat accumulation in myocytes cultured in vitro, e.g., with primary human differentiated myoblasts in medium designed to replicate a sarcopenic plasma profile and tested for lipid accumulation, e.g., as described in Example 2.

Broadly, the invention includes a method for improving muscle function in reduced-mobility or immobilized muscle by reducing fat infiltration, wherein the method comprises administering to a subject in need thereof an effective amount of a composition (e.g., an Active Moiety), wherein the composition reduces fat infiltration in muscle, e.g., in the reduced-mobility or immobilized muscle. This method can be practiced with a composition that is a pharmaceutical composition. Thus, the composition can further comprise a pharmaceutically acceptable excipient, such as an excipient that is suitable for oral administration. The discovery that such compositions are capable of reducing infiltration of fat in muscle thus allows for determining a degree of fat infiltration in muscle under conditions of reduced-mobility or immobilized muscle. Any method for determining or evaluating the degree of fat infiltration in muscle can be used, e.g., MRI, DEXA, or CT.

In these foregoing methods for improving muscle function in reduced-mobility or immobilized muscle by reducing fat infiltration, the subject can have a disease or disorder selected from the group consisting of a rare muscle disease, muscle atrophy, cirrhosis with or without sarcopenia, muscle deterioration, muscle decay, cachexia, drug-induced myopathy, muscular dystrophy, myopenia, muscle weakness, perceived muscle weakness, ICU-acquired myopathy, burns-related myopathy, a neuromuscular disorder, ventilator-induced diaphragmatic dystrophy, hyponatremia, hypokalemia, a calcium deficiency, hypercalcemia, amyotrophic lateral sclerosis, and a bone weakness disease. Alternatively, the subject can have or be identified as having decreased muscle function due to aging, injury, muscle atrophy, infection, disease, stroke, a neurological injury, or a fracture or other trauma. The fracture or other trauma may be selected from rotator cuff surgery, knee surgery, hip surgery, joint replacement, injury repair surgery, or the subject has worn a cast. In the case of a fracture or trauma, the subject can receive the composition after the fracture or other trauma or before the fracture or other trauma, in the latter case, e.g., in conjunction with planned elective surgery. In yet another embodiment, the subject at risk of fat infiltration in muscle has cancer, e.g., colorectal cancer or periampullary cancer. Preferably the cancer is treated surgically in conjunction with the methods of the invention.

In some embodiments, the method can be used to reduce fat infiltration in muscle of a subject at risk of fat infiltration in muscle who has a rotator cuff injury, and in particular, administration of the composition precedes a surgery for the rotator cuff injury. The invention can include determining a level of fat infiltration in shoulder muscle affected by the rotator cuff injury, e.g., before surgery or after surgery. In a specific embodiment, the subject with a rotator cuff injury is an elderly subject.

In some embodiments, the subject at risk for fat infiltration in muscle does not have significant increase in BMI, sarcopenia, or other overt conditions, or the subject at risk for fat infiltration in muscle suffers from cirrhosis without sarcopenia.

In some embodiments, administering the composition of the invention (e.g., Active Moiety) to the subject at risk of fat infiltration in muscle improves a muscle function of sequestering glucose. For example, and not by way of limitation, the subject at risk for fat infiltration in muscle has diabetes or metabolic disease. Alternatively, the subject at risk for fat infiltration in muscle may suffer from cirrhosis with sarcopenia or cirrhosis without sarcopenia. In another aspect, the subject has or is at risk for chronic back pain (e.g., fat infiltration in paraspinal muscles); HIV patients (e.g., fat infiltration in locomotor muscles); spinal cord injury; stroke; COPD; end-stage liver disease (ESLD) (e.g., the subject has one or more of hepatic encephalopathy, variceal bleeding, portal hypertension, ascites, infection risk, sepsis, all-cause hospitalization, and all-cause and liver-related mortality); and muscle weakness associated with ageing. Furthermore, the administering the composition to the subject at risk of fat infiltration in muscle can improve a muscle function of sequestering glucose, e.g., when the subject at risk for fat infiltration in muscle has diabetes or metabolic disease.

The invention also provides for determining or evaluating fat infiltration in muscle in the subject to evaluate effectiveness of administration of the composition in treating the disease or disorder, decreased muscle function, condition involving fat infiltration in muscle, fracture or other trauma, e.g., prior to an elective procedure. As exemplified herein, this method can comprise evaluating the ability of the composition to reduce fat accumulation in myocytes cultured in vitro, e.g., with primary human differentiated myoblasts in medium designed to replicate a sarcopenic plasma profile and tested for lipid accumulation, e.g., as described in Example 2.

Accordingly, in one aspect, the invention provides a method for determining whether a composition comprising a histidine amino acid entity, a lysine amino acid entity, and a threonine amino acid entity is effective in treating a disease or disorder associated with muscle function. This method comprises administering to the subject a composition (e.g., Active Moiety) comprising a histidine amino acid entity, a lysine amino acid entity, and a threonine amino acid entity and determining whether there is a reduction in fat infiltration in muscle in the subject. In one embodiment, fat infiltration is in muscle tissue affected by the disease or disorder associated with muscle function. According to the invention and exemplified below, determining or evaluating fat infiltration in muscle reveals that a fat fraction in muscle is unchanged from before the treatment, or even improved. As noted above, any method for determining or evaluating the degree of fat infiltration in muscle can be used, e.g., MRI, DEXA, or CT.

Thus, fat infiltration in muscle can serve as a surrogate for muscle function in a study, e.g., a clinical trial, post-marketing trial, prognostic assay, etc. in conjunction with treatment with a composition described herein, e.g., comprising a histidine amino acid entity, a lysine amino acid entity, and a threonine amino acid entity. In a specific embodiment, in the situation where the subject has has a rotator cuff injury, the method can be used. For example, administration of the composition can precede a surgery for the rotator cuff injury. In some embodiments, the invention provides for determining a level of fat infiltration in shoulder muscle affected by the rotator cuff injury before surgery. In conjunction with evaluating progression and prognosis, it is also possible to determine a level of fat infiltration in shoulder muscle affected by the rotator cuff injury after surgery. The methods of the invention can further comprise determining a level of fat infiltration in thigh muscle. In a specific embodiment, the degree of fat infiltration in muscle is determined by magnetic resonance imaging (MRI).

### BRIEF DESCRIPTION OF THE DRAWINGS

FIG. 1 is a schematic illustrating the study design.

### DETAILED DESCRIPTION

The present invention is based on the discovery that compositions comprising amino acid entities are capable of reducing fat infiltration in muscle. This discovery provides for treatment of a number of diseases and disorders involving fat infiltration in muscle. In some cases, fat infiltration in muscle results in increased morbidity, worsening of the disease or disorder, or predicts a worse outcome from an intervention, e.g., to treat a disease or disorder of the liver, such as cirrhosis, hepatic encephalopathy, and cirrhotic sarcopenia.

Thus the invention provides, at least in part, methods of reducing fat infiltration in muscle by administering a composition, e.g., Active Moiety, of the invention, which is a composition comprising at least three different amino acid entities (e.g., a histidine amino acid entity, a lysine amino acid entity, and a threonine amino acid entity). In some embodiments, the composition, e.g., Active Moiety, does not include a physiologically effective amount of one or more of leucine, isoleucine, valine, glutamine, arginine, and N-acetylcysteine (e.g., one or more of leucine, isoleucine, valine, glutamine, arginine, and N-acetylcysteine is absent or, if present, is present at less than: 1 wt. %, 0.5 wt. %, 0.1 wt. %, 0.05 wt. %, 0.01 wt. %, 0.001 wt. %, or less, e.g., of the total wt. of the composition (e.g., in dry form). In some cases, the invention further comprises determining or evaluating the extent or degree of infiltration of fat in muscle, e.g., as a diagnostic, a prognostic indicator, to evaluate progress of the disease or disorder with or without treatment, or as a surrogate of muscle health, or any combination of the foregoing. Various techniques are available for determining the extent of infiltration of fat in muscle. The most rigorous techniques are computed tomograph (CT) and magnetic resonance imaging (MRI).

The methods of reducing fat infiltration in muscle may also provide a method of treating any or all of immobilization, malnutrition, fasting, aging, autophagy, reduced protein synthesis, anabolic resistance, junction integrity (e.g., neuromuscular junction integrity), insulin resistance, decreased mitochondrial biogenesis, an energy deficit, or anaplerosis in a subject that includes administering to a subject in need thereof an effective amount of a pharmaceutical composition including defined amino acid components. In some embodiments, the subject has a rare muscle disease. In some embodiments, the subject has sarcopenia, muscle deterioration, decay, atrophy, cachexia, drug-induced myopathy, muscular dystrophy, or myopenia. In some embodiments, the subject has a fracture or other trauma. In some embodiments, the subject has a drug-induced myopathy. In some embodiments, the subject has a statin-induced myopathy. In some embodiments, the subject has a steroid-induced myopathy. In some embodiments, the subject has an immunosuppressant-induced myopathy. In some embodiments, the subject has a chemotherapeutic-induced myopathy. In some embodiments, the subject has an alcohol-induced myopathy.

In addition to evaluating or determining the extent of fat infiltration in muscle, improvements in muscle function can be assessed by performing metrics selected from maximal isometric knee strength test (e.g., to determine changes in muscle strength), muscle biopsy (e.g., to determine muscle fiber quality), and electrical impedance myography (EIM) (e.g., to determine muscle health, such as resistive and capacitive properties of muscle tissue and sensitivity to disuse-related atrophy), or other standard clinical performance assessments such as the Short Performance Physical Battery (SPPB), Harris Hip Score and others.

In some embodiments, the composition is for use as a medicament in improving muscle function in a subject at risk of or experiencing fat infiltration in muscle. In some embodiments, the composition is for use as a medicament in treating a muscle disease or disorder involving fat infiltration in the muscle in a subject.

In some embodiments, the composition is for use in the manufacture of a medicament for improving muscle function in a subject. at risk of or experiencing fat infiltration in muscle. In some embodiments, the composition is for use in the manufacture of a medicament for treating a muscle disease or disorder involving fat infiltration in the muscle in a subject.

Additionally, the composition may be useful as a dietary supplement, e.g., a nutritional supplement, dietary formulation, functional food, medical food, food, or beverage comprising a composition described herein. Another embodiment provides a nutritional supplement, dietary formulation, functional food, medical food, food, or beverage comprising a composition described herein for use in the management of any of the diseases or disorders described herein.

One embodiment provides a method of maintaining or improving muscle health, muscle function, muscle functional performance, or muscle strength, comprising administering to a subject an effective amount of a composition described herein to reduce fat infiltration in muscle in the subject. Another embodiment provides a method of providing nutritional support or supplementation to a subject suffering from muscle atrophy comprising administering to the subject an effective amount of a composition described herein to reduce fat infiltration in muscle in the subject. Yet another embodiment provides a method of providing nutritional support or supplementation that aids in the management of muscle atrophy to a subject comprising administering to the subject in need thereof an effective amount of a composition described herein to reduce fat infiltration in muscle in the subject.

### Definitions

Terms used in the claims and specification are defined as set forth below unless otherwise specified.

It must be noted that, as used in the specification and the appended claims, the singular forms “a,” “an” and “the” include plural referents unless the context clearly dictates otherwise.

“About” and “approximately” shall generally mean an acceptable degree of error for the quantity measured given the nature or precision of the measurements. Exemplary degrees of error are within 20 percent (%), typically, within 10%, and more typically, within 5% of a given value or range of values.

The term “fat infiltration in muscle” means an increase in the fat fraction of muscle below the muscle fascia, as distinguished from subcutaneous fat. For example, fat found in the deep fascia of the thigh is fat infiltration in muscle. Various publications use terms such as intramuscular fat fraction, intermuscular fat infiltration, intramuscular fat, intermuscular fat, intermuscular adipose tissue (IMAT), and myosteatosis or myostasis. All of these terms refer to fat (visible storage of lipids in adipocytes) located between muscle fibers and between muscle groups, or within myocytes themselves. Fat that has infiltrated in muscle appears to have some of the same characteristic as ectopic or visceral fat, e.g., fat in liver or other organs, or in the abdomen.

“Reduction of fat infiltration in muscle” means that the degree or extent of fat infiltration in a subject, e.g., to muscle that is immobilized, injured, or otherwise subject to muscle infiltration, is less than it would have been in the absence of an intervention, i.e., in the absence of administering an Active Moiety to the subject. Thus, the fat fraction in the muscle is lower than it would have otherwise been. In one example, administration of an Active Moiety reduces fat infiltration in muscle by 100%., e.g., it prevents it. However, the invention includes any reduction in fat infiltration that would occur in the absence of treatment with an Active Moiety. Preferably reduction of fat infiltration in muscle is detectable. More preferably it is significant, e.g., reaches statistical significance in a population of subjects in a controlled study.

The term “fat fraction” or “FF” refers to the fraction of fat or percentage of fat in a limb, part of a limb, or body (taking the entire volume of the limb, part of a limb, or body as the whole or 100%). Similarly, the muscle mass is the fraction of muscle or percentage of muscle in limb, part of a limb, or body.

Images depicting fat fraction (FF) show that in a subject administered placebo, the subject had increased FF and decreased muscle mass. By contrast, in a subject administered an Active Moiety, the muscle had lower fat fraction and a higher muscle content compared to placebo.

The term “subject” refers to a human person, and can include, but is not limited to, a patient, i.e., a person who is under care of a healthcare provider (doctor, nurse practitioner, etc.). It can also mean a person in a clinical study, or a person who self-diagnoses and self-treats, or a subject who receives a dietary supplement (as broadly defined above).

As used herein, the term “Active Moiety” means a combination comprising three or more amino acid entities that, in aggregate, has a physiological effect. In such a case, the amino acid entity is present in a “physiologically effective amount,” which is an amount effective to produce a physiological effect. In some cases, the physiological effect can be a therapeutic effect involving reduction of fat infiltration into muscle, as defined below. For example, an Active Moiety can rebalance a metabolic dysfunction in a subject suffering from a disease or disorder. An Active Moiety of the invention can contain other biologically active ingredients. In some examples, the Active Moiety comprises a histidine amino acid entity, a lysine amino acid entity, and a threonine amino acid entity, and does not include a physiologically effective amount of one or more of leucine, isoleucine, valine, glutamine, arginine, and N-acetylcysteine, as set out in detail below (e.g., one or more of leucine, isoleucine, valine, glutamine, arginine, and N-acetylcysteine is absent or, if present, is present at less than: 1 wt. %, 0.5 wt. %, 0.1 wt. %, 0.05 wt. %, 0.01 wt. %, 0.001 wt. %, or less, e.g., of the total wt. of the composition (e.g., in dry form). An Active Moiety of the invention can contain other biologically active ingredients.

The individual amino acid entities are present in the Active Moiety in various amounts or ratios, which can be presented as amount by weight (e.g., in grams), ratio by weight of amino acid moieties to each other, amount by mole, amount by dry weight percent of the Active Moiety, amount by mole percent of the Active Moiety, caloric content, percent caloric contribution to the Active Moiety, etc. Generally, this disclosure will provide grams of amino acid entity in a dosage form, weight percent of an amino acid moiety relative to the weight of the Active Moiety, i.e., the weight of all the amino acid moieties and any other biologically active ingredient present in the Active Moiety, or in ratios. The abbreviation “wt.” means weight. Where an amino acid entity is not present “in a physiologically effective amount” that means if it present, it has no physiological effect. In a specific embodiment, such an amount is less than 1% of the total weight of amino acids in the Active Moiety. In a further embodiment, such an amount cannot be measured by standard analytical techniques typically used in the pharmaceutical industry. In a further embodiment, such an amount is the total absence of the amino acid entity.

As used herein, the term “amino acid entity” refers to an amino acid in one or both of free form or salt form, an amino acid residue of a peptide (e.g., of a dipeptide, tripeptide, oligopeptide, or polypeptide of 20 amino acids or less in length), a derivative of an amino acid, a precursor of an amino acid, or a metabolite of an amino acid (see, e.g., Table 1). Accordingly, the term “XXX amino acid entity” refers to an amino acid entity that if a free amino acid, comprises free XXX or XXX in salt form; if a peptide, refers to a peptide of fewer than 20 amino acids, and preferably two or three amino acids, comprising an XXX residue; if a derivative, refers to a derivative of XXX; if a precursor, refers to a precursor of XXX; and if a metabolite, refers to a XXX metabolite. Where the biological system provides for isomerization of a D-amino acid to the L-form, the D-amino acid can be an amino acid entity. For example, where XXX is leucine (L), then leucine amino acid entity refers to free L or L in salt form, a peptide (e.g., a dipeptide or a tripeptide) comprising a L residue, a L derivative, a L precursor, or a metabolite of L; where XXX is isoleucine (I), then isoleucine amino acid entity refers to free I or I in salt form, a peptide (e.g., a dipeptide or a tripeptide) comprising a I residue, a I derivative, a I precursor, or a metabolite of I; where XXX is valine (V), then valine amino acid entity refers to free V or V in salt form, a peptide (e.g., a dipeptide or a tripeptide) comprising a V residue, a V derivative, a V precursor, or a metabolite of V; where XXX is ornithine (Orn), then ornithine amino acid entity refers to free Orn or Orn in salt form, a peptide (e.g., a dipeptide or a tripeptide) comprising a Orn residue, a Orn derivative, a Orn precursor, or a metabolite of Orn; where XXX is aspartate (D), then aspartate amino acid entity refers to free D or D in salt form, a peptide (e.g., a dipeptide or a tripeptide) comprising a D residue, a D derivative, a D precursor, or a metabolite of D; where XXX is histidine (H), then histidine amino acid entity refers to free H or H in salt form, a peptide (e.g., a dipeptide or a tripeptide) comprising a H residue, a H derivative, a H precursor, or a metabolite of H; where XXX is lysine (K), then lysine amino acid entity refers to free K or K in salt form, a peptide (e.g., a dipeptide or a tripeptide) comprising a K residue, a K derivative, a K precursor, or a metabolite of K; and where XXX is threonine (T), then threonine amino acid entity refers to free T or T in salt form, a peptide (e.g., a dipeptide or a tripeptide) comprising a T residue, a T derivative, a T precursor, or a metabolite of T.

Salts of amino acids include any physiologically tolerable, e.g., ingestible, salt. For pharmaceutical compositions, the salt form of an amino acid present in the Active Moiety should be a pharmaceutically acceptable salt. In a specific example, the salt form is the hydrochloride (HCl) salt form of the amino acid.

In some embodiments, the derivative of an amino acid entity comprises an amino acid ester (e.g., an alkyl ester, e.g., an ethyl ester or a methyl ester of an amino acid entity) or a keto-acid.

TABLE 1
Amino add entities include amino adds, precursors, metabolites,
and derivatives of the compositions described herein.

| | Exemplary | | | | |
|---|---|---|---|---|---|
| | Amino Acid | Precursors | Metabolites | Derivatives | Salts |
| Leucine | L-Leucine | Oxo-leucine | HMB (beta- | N-Acetyl- | |
| | | (Alpha- | hydroxy-beta- | Leucine | |
| | | ketoisocaproate | methybutyrate); | | |
| | | (KIC)) | Oxo-leucine; | | |
| | | | Isovaleryl-CoA | | |
| Isoleucine | L-Isoleucine | 2-Oxo-3-methyl- | 2-Oxo-3-methyl- | N-Acetyl- | |
| | | valerate (Alpha- | valerate; | Isoleucine | |
| | | keto-beta- | Methylbutyrl-CoA | | |
| | | methylvaleric | | | |
| | | acid (KMV)); | | | |
| | | Threonine | | | |
| Valine | L-Valine | 2-Oxo-valerate | Isobutryl-CoA | N-Acetyl- | |
| | | (alpha- | | Valine | |
| | | ketoisovalerate | | | |
| | | (KIV) | | | |
| Ornithine | L-Ornithine | L-Arginine, | Citrulline | | Ornithine |
| | | Glycine | | | α- |
| | | | | | ketoglutarate, |
| | | | | | Ornithine |
| | | | | | HCl |
| Arginine | L-Arginine | Arginosuccinate; | Agmatine; | N-Acetyl- | |
| | | Aspartate; | Creatine | Arginine; | |
| | | Glutamate | | | |
| Aspartate | L-Aspartate | Fumarate | Adenylosuccinate | | |
| NAC | N- | Acetylserine; | Glutathione; | Cystine; | |
| | Acetylcysteine | Cystathionine; | Cystathionine; | Cysteamine | |
| | | | Homocysteine; | | |
| | | | Methionine | | |
| Histidine | L-Histidine | Histidinol; | Carnosine; | N-Acetyl- | |
| | | Histidinal; | Histamine; | Histidine | |
| | | Ribose-5- | Urocanate | | |
| | | phosphate | | | |
| Lysine | L-Lysine | Diaminopimelate; | Trimethylhistidine | N-Acetyl- | L- |
| | | Aspartate | amino acid entity; | Lysine | Lysine |
| | | | Trimethyllyisine; | | Acetate |
| | | | Carnitine; | | |
| | | | Saccharopine | | |
| Phenylalanine | L- | Phenylpyruvate | Tyrosine | N-Acetyl- | |
| | Phenylalanine | | | Phenylalanine | |
| Threonine | L-Threonine | Homoserine; O- | Oxobutyrate | N-Acetyl- | |
| | | PhosphoHomoserine | | Threonine | |

An “amino acid” refers to an organic compound having an amino group (—NH₂), a carboxylic acid group (—C(═O)OH), and a side chain bonded through a central carbon atom, and includes essential and non-essential amino acids, as well as natural and unnatural amino acids. Unless otherwise indicated, amino acids referred to herein are L-isomers of amino acids.

The proteogenic amino acids, shown below, are known by three- and one-letter abbreviations in addition to their full names. For a given amino acid, these abbreviations are used interchangeably herein. For example, Leu, L or leucine all refer to the amino acid leucine; Ile, I or isoleucine all refer to the amino acid isoleucine; Val, V or valine all refer to the amino acid valine; Arg, R or arginine all refer to the amino acid arginine; and Gln, Q or glutamine all refer to the amino acid glutamine. Likewise, the non-natural amino acid derivative N-acetylcysteine may be referred to interchangeably by “NAC” or “N-acetylcysteine.” Amino acids may be present as L-isomers of amino acids to ensure physiological activity.

TABLE 2
Canonical (proteogenic) amino acid names and abbreviations

| Amino acid (L isomer) | Three-letter | One-letter |
|---|---|---|
| Alanine | Ala | A |
| Arginine | Arg | R |
| Asparagine | Asn | N |
| Aspartic acid | Asp | D |
| Cysteine | Cys | C |
| Glutamic acid | Glu | E |
| Glutamine | Gln | Q |
| Glycine | Gly | G |
| Histidine | His | H |
| Isoleucine | Ile | I |
| Leucine | Leu | L |
| Lysine | Lys | K |
| Methionine | Met | M |
| Phenylalanine | Phe | F |
| Proline | Pro | P |
| Serine | Ser | S |
| Threonine | Thr | T |
| Tryptophan | Trp | W |
| Tyrosine | Tyr | Y |
| Valine | Val | V |

A “branched chain amino acid” is an amino acid selected from leucine, isoleucine, and valine. A “aromatic amino acid” is an amino acid selected from phenylalanine, tryptophan, and tyrosine.

The term “effective amount” as used herein means an amount of a composition, e.g., Active Moiety, or pharmaceutical composition comprising an Active Moiety, which is sufficient to significantly and positively modify the symptoms and/or conditions to be treated (e.g., provide a positive clinical response). The effective amount of an active ingredient for use in a pharmaceutical composition will vary with the particular condition being treated, the severity of the condition, the duration of treatment, the nature of concurrent therapy, the particular active ingredient(s) being employed, the particular pharmaceutically-acceptable excipient(s) and/or carrier(s) utilized, and like factors with the knowledge and expertise of the attending physician.

An “equivalent amount” of an amino acid entity is an amount that yields, physiologically, the same activity as that amount of the corresponding free amino acid for the amino acid entity. A “pharmaceutical composition” described herein comprises at least one amino acid or

Active Moiety and a pharmaceutically acceptable carrier or excipient. In some embodiments, the pharmaceutical composition is used as a therapeutic. Other compositions, which need not meet pharmaceutical standards (GMP; pharmaceutical grade components) can be used as a nutraceutical, a medical food, or as a supplement, these are termed “consumer health compositions.”

The term “pharmaceutically acceptable” as used herein, refers to amino acids, materials, excipients, compositions and/or dosage forms which are, within the scope of sound medical judgment, suitable for use in contact with the tissues of human beings and animals without excessive toxicity, irritation, allergic response, or other problem or complication, commensurate with a reasonable benefit/risk ratio. In a specific embodiment, “pharmaceutically acceptable” means free of detectable endotoxin or endotoxin levels are below levels acceptable in pharmaceutical products. In a specific embodiment, “pharmaceutically acceptable” means a standard used by the pharmaceutical industry or by agencies or entities (e.g., government or trade agencies or entities) regulating the pharmaceutical industry to ensure one or more product quality parameters are within acceptable ranges for a medicine, pharmaceutical composition, treatment, or other therapeutic. A product quality parameter can be any parameter regulated by the pharmaceutical industry or by agencies or entities, e.g., government or trade agencies or entities, including but not limited to composition; composition uniformity; dosage; dosage uniformity; presence, absence, and/or level of contaminants or impurities; and level of sterility (e.g., the presence, absence and/or level of microbes). Exemplary government regulatory agencies include: Federal Drug Administration (FDA), European Medicines Agency (EMA), SwissMedic, China Food and Drug Administration (CFDA), or Japanese Pharmaceuticals and Medical Devices Agency (PMDA).

The term “pharmaceutically acceptable excipient” refers to an ingredient in a pharmaceutical formulation, other than an active, which is physiologically compatible. A pharmaceutically acceptable excipient can include, but is not limited to, a buffer, a sweetener, a dispersion enhancer, a flavoring agent, a bitterness masking agent, a natural coloring, an artificial coloring, a stabilizer, a solvent, or a preservative. In a specific embodiment, a pharmaceutically acceptable excipient includes one or both of citric acid or lecithin.

The term “non-amino acid entity protein component,” as used herein, refers to a peptide (e.g., a polypeptide or an oligopeptide), a fragment thereof, or a degraded peptide Exemplary non-amino acid entity protein components include, but are not limited to, one or more of whey protein, egg white protein, soy protein, casein, hemp protein, pea protein, brown rice protein, or a fragment or degraded peptide thereof.

The term “non-protein component,” as used herein, refers to any component of a composition other than a protein component. Exemplary non-protein components can include, but are not limited to, a saccharide (e.g., a monosaccharide (e.g., dextrose, glucose, or fructose), a disaccharide, an oligosaccharide, or a polysaccharide); a lipid (e.g., a sulfur-containing lipid (e.g., alpha-lipoic acid), a long chain triglyceride, an omega 3 fatty acid (e.g., EPA, DHA, STA, DPA, or ALA), an omega 6 fatty acid (GLA, DGLA, or LA), a medium chain triglyceride, or a medium chain fatty acid); a vitamin (e.g., vitamin A, vitamin E, vitamin C, vitamin D, vitamin B6, vitamin B12, biotin, or pantothenic acid); a mineral (zinc, selenium, iron, copper, folate, phosphorous, potassium, manganese, chromium, calcium, or magnesium); or a sterol (e.g., cholesterol).

A “therapeutic effect” means a beneficial clinical effect. A beneficial clinical effect can be shown by lessening the progression of a disease and/or alleviating one or more symptoms of the disease. Preferably, the beneficial clinical effect is statistically significant.

A “health effect” in the context of a food supplement means that there is some benefit to the normal structure or function of a human or target organ in a human.

A “unit dose” or “unit dosage” as used herein means an amount or dose of medicine prepared in an individual packet or container for convenience, safety, or monitoring. A “unit dose” or “unit dosage” comprises the drug product or drug products in the form in which they are marketed for use, with a specific mixture of active ingredients and inactive components (excipients), in a particular configuration (e.g., a capsule shell, for example), and apportioned into a particular dose (e.g., in multiple stick packs).

As used herein, the terms “treat,” “treating,” or “treatment” refer in one embodiment, to ameliorating a disease or disorder of fat infiltration in muscle (i.e., slowing or arresting or reducing the development of the disease or disorder or at least one of the clinical symptoms thereof). In another embodiment, “treat,” “treating,” or “treatment” refers to alleviating or ameliorating at least one physical parameter of fat infiltration in muscle, including those which may not be discernible by the patient. In yet another embodiment, “treat,” “treating,” or “treatment” refers to modulating a symptom of a disease or disorder of fat infiltration in muscle. In yet another embodiment, “treat,” “treating,” or “treatment” refers to preventing or delaying the onset or development or progression of a disease, disorder, or condition involving fat infiltration in muscle.

### Active Moieties: Compositions of Amino Acid Entities

US Patent Publication No. 2019/0046487 A1, published Feb. 14, 2019, entitled “COMPOSITIONS AND METHODS FOR TREATMENT OF LIVER DISEASES AND DISORDERS ASSOCIATED WITH ONE OR BOTH OF HYPERAMMONEMIA OR MUSCLE WASTING,” which is specifically incorporated herein by reference in its entirety, disclose compositions of amino acid entities, e.g., Active Moieties, which may reduce fat infiltration in muscle, as can be shown by the methods herein described. U.S. patent application Ser. No. 16/446,328, filed Jun. 19, 2019, entitled “COMPOSITIONS AND METHODS FOR THE TREATMENT OF FAT INFILTRATION IN MUSCLE,” which is specifically incorporated herein by reference in its entirety, discloses compositions of amino acid entities, e.g., Active Moieties, that are shown to reduce fat infiltration in muscle.

In some embodiments, the composition comprises one or more of a histidine (H)-amino acid entity, a lysine (K)-amino acid entity, and a threonine (T)-amino acid entity. In a further embodiment, for subject who do not have elevated levels of serum phenylalanine, the composition also comprises a phenylalanine (F)-amino acid entity, with the proviso that the composition is not a composition of all essential amino acids (EAAs). In all embodiments, the composition does not include a physiologically effective amount of an amino acid selected from leucine, isoleucine, valine, glutamine, arginine, and N-acetylcysteine.

In some embodiments, the total weight of the histidine (H)-amino acid entity, the lysine (K)-amino acid entity, and the threonine (T)-amino acid entity, and optionally the phenylalanine (F)-amino acid entity, can be greater than the total weigh. of other amino acid entities in the composition (e.g., Active Moiety).

The weight ratio of a particular amino acid or particular amino acids in a composition or mixture of amino acids, e.g., an Active Moiety, is the ratio of the weight of the particular amino acid or amino acids in the composition or mixture compared to the total weight of amino acids present in the composition or mixture. This value is calculated by dividing the weight of the particular amino acid or of the particular amino acids in the composition or mixture by the weight of all amino acids present in the composition or mixture and multiplying times 100. Percent weight on a dry weight basis refers to the percent weight of solid materials, which is relevant because as exemplified herein the Active Moiety composition may be dissolved or suspended in a liquid, particularly water, which provides considerable additional weight to a final liquid formulation.

The composition, e.g., Active Moiety may further comprise additional branched-chain amino acid (BCAA)-entities, e.g., one, both, or all three of a leucine amino acid entity, an isoleucine amino acid entity, and a valine amino acid entity; alternatively, both the isoleucine amino acid entity and the valine amino acid entity are present.

Preferably, at least one amino acid entity is a free amino acid, e.g., one or more (e.g., all) amino acid entities are a free amino acid. In some embodiments, each of one or more of the histidine (H)-amino acid entity, the lysine (K)-amino acid entity, and the threonine (T)-amino acid entity, and optionally the phenylalanine (F)-amino acid entity, is a free amino acid entity.

Alternatively, at least one amino acid entity is in a salt form, e.g., one or more (e.g., all) of the amino acid entities is in a salt form. In some embodiments, each of one or more of the histidine (H)-amino acid entity, the lysine (K)-amino acid entity, and the threonine (T)-amino acid entity, and optionally the phenylalanine (F)-amino acid entity, is in a salt form.

In some embodiments, the composition, e.g., Active Moiety, comprises a combination of 3 to 20 different amino acid entities, e.g., 4 to 15 different amino acid entities. In other embodiments, the Active Moiety consists of 3 to 16 different amino acid entities; more particularly, 5 to 15 different amino acid entities.

In some embodiments, the composition further comprises one or more of arginine, glutamine, N-acetylcysteine; a BCAA chosen from one, two, or all of leucine, isoleucine, and valine; provided that the composition does not include a physiologically effective amount of an amino acid selected from leucine, isoleucine, valine, glutamine, arginine, and N-acetylcysteine, and the composition is not a composition of all essential amino acids (EAAs).

In some embodiments, the composition described herein (e.g., an Active Moiety) comprises amino acid entities, e.g., the amino acid entities shown in Table 1.

In certain embodiments, the leucine amino acid entity is chosen from Table 1, e.g., the leucine amino acid entity is chosen from L-leucine, β-hydroxy-β-methylbutyrate (HMB), oxo-leucine (alpha-ketoisocaproate (KIC)), isovaleryl-CoA, n-acetylleucine, or a combination thereof. In certain embodiments, the leucine amino acid entity is chosen from L-leucine, oxo-leucine (KIC), isovaleryl-CoA, n-acetyl-leucine, or a combination thereof.

In certain embodiments, the isoleucine amino acid entity is chosen from Table 1, e.g., the isoleucine amino acid entity is chosen from L-isoleucine, 2-oxo-3-methyl-valerate (alpha-keto-beta-methylvaleric acid (KMV)), threonine, methylbutyryl-CoA, D-isoleucine, N-acetyl-isoleucine, or a combination thereof.

In certain embodiments, the valine amino acid entity is chosen from Table 1, e.g., the valine amino acid entity is chosen from L-valine, 2-oxo-valerate (alpha-ketoisovalerate (KIV)), isobutyryl-CoA, N-acetyl-valine, or a combination thereof.

In certain embodiments, the ornithine amino acid entity is chosen from Table 1, e.g., the ornithine amino acid entity is chosen from L-ornithine, ornithine α-ketoglutarate, ornithine HCl, L-arginine, glycine, citrulline, or a combination thereof. In certain embodiments, the ornithine amino acid entity is chosen from L-ornithine, ornithine α-ketoglutarate, ornithine HCl, citrulline, or a combination thereof. In certain embodiments, the ornithine amino acid entity is chosen from L-ornithine, ornithine HCl, citrulline, or a combination thereof.

In certain embodiments, the aspartate amino acid entity is chosen from Table 1, e.g., the aspartate amino acid entity is chosen from L-aspartate, fumarate, adenylosuccinate, or a combination thereof.

In certain embodiments, the histidine amino acid entity is chosen from Table 1, e.g., the histidine amino acid entity is chosen from L-histidine, histidinol, histidinal, ribose-5-phosphate, carnosine, histamine, urocanate, and N-acetyl-histidine, or a combination thereof.

In certain embodiments, the lysine amino acid entity is chosen from Table 1, e.g., the lysine amino acid entity is chosen from L-lysine, diaminopimelate, aspartate, trimethylhistidine amino acid entity, carnitine, saccharopine, N-acetyl-lysine, or a combination thereof.

In certain embodiments, the threonine amino acid entity is chosen from Table 1, e.g., the threonine amino acid entity is chosen from L-threonine, homoserine, O-phosphohomoserine, oxobutyrate, N-acetyl-threonine, or a combination thereof.

In some embodiments, one, two, or three of (a) a leucine amino acid entity, an isoleucine amino acid entity, or a valine amino acid entity is in free amino acid form. In some embodiments, one, two, or three of (a) a leucine amino acid entity, an isoleucine amino acid entity, a valine amino acid entity is in salt amino acid form.

In some embodiments, one or both of (b) an ornithine amino acid entity or an aspartate amino acid entity is in free amino acid form. In some embodiments, one or both of (b) ornithine amino acid entity or an aspartate amino acid entity is in salt amino acid form (e.g., L-ornithine or a salt thereof and L-aspartate or a salt thereof are present in combination as a salt (LOLA)).

In some embodiments, one, two, or three of (c) a histidine amino acid entity, a lysine amino acid entity, or a threonine amino acid entity is in free amino acid form. In some embodiments, one, two, or three of (c) a histidine amino acid entity, a lysine amino acid entity, or a threonine amino acid entity is in salt amino acid form (e.g., L-lysine or a salt thereof is present as L-lysine acetate).

In some embodiments, at least: 35 wt. %, 40 wt. %, 42 wt. %, 45 wt. %, 50 wt. %, 55 wt. %, 60 wt. %, 70 wt. %, 80 wt. %, or more, of the total wt. of the composition (in dry form) is three, four, five, six, seven, or eight amino acid entities in (a)-(c) or (i)-(iii) in free amino acid form. In some embodiments, at least: 15 wt. %, 20 wt. %, 25 wt. %, 35 wt. %, 40 wt. %, or more, of the total wt. of the composition (in dry form) is three, four, five, six, seven, or eight amino acid entities in (a)-(c) or (i)-(iii) in salt form.

In some embodiments, three, four, five, six, seven, or eight amino acid entities in (a)-(c) or or (i)-(iii) is provided as part of a dipeptide or tripeptide, e.g., in an amount of at least: 0.01 wt. %, 0.1 wt. %, 0.5 wt. %, 1 wt. %, 5 wt. %, or 10 wt. %, or more of amino acid entities or total components of the composition.

In some embodiments, the composition further comprises L-alanine, L-arginine, L-tryptophan, carnitine, sodium acetate, or a combination thereof. In some embodiments, the composition further comprises a mineral, e.g., zinc. In some embodiments, the composition further comprises a vitamin, e.g., one, two, or three of vitamin A, vitamin I), vitamin E, or a combination thereof. In some embodiments, the composition further comprises an ammonia scavenger, e.g., phenyl acetate, acetyl-L-carnitine, citrulline, sodium benzoate, sodium phenylbutyrate, or a combination thereof.

In some embodiments, the composition can include sulfur AAs (SAAs), such as N-acetylcysteine (NAC). In an embodiment, the SAA (e.g., NAC) has anti-oxidant activity. In an embodiment, the SAA (e.g., NAC) results in decreased reactive oxygen species (ROS) or increased glutathione (GSH) in a subject administered the composition described herein.

In some embodiments, the composition comprises, consists of, or consists essentially of: a leucine amino acid entity, an isoleucine amino acid entity, valine amino acid entity, an ornithine amino acid entity, an aspartate amino acid entity, a histidine amino acid entity, a threonine amino acid entity, and a lysine amino acid entity.

In some embodiments, the composition (e.g., the Active Moiety) comprises, consists of, or consists essentially of: a) a leucine amino acid entity; b) an ornithine amino acid entity; and c) an essential amino acid (EAA)-entity chosen from a histidine amino acid entity, a lysine amino acid entity, or a threonine amino acid entity or a combination of two or three EAA entities; wherein at least one amino acid entity (e.g., two, three, four, or five amino acid entities) of (a)-(c) is not provided as a peptide of more than 20 amino acid residues in length.

In some embodiments, the composition (e.g., the Active Moiety) comprises, consists of, or consists essentially of: a) a leucine amino acid entity and a valine amino acid entity; b) an ornithine amino acid entity; and c) an essential amino acid (EAA)-entity chosen from a histidine amino acid entity, a lysine amino acid entity, or a threonine amino acid entity or a combination of two or three EAA entities; wherein at least one amino acid entity (e.g., two, three, four, or five amino acid entities) of (a)-(c) is not provided as a peptide of more than 20 amino acid residues in length. In some embodiments, the composition further comprises an isoleucine amino acid entity. In some embodiments, the composition further comprises an aspartate amino acid entity.

In some embodiments, the composition (e.g., the Active Moiety) comprises, consists of, or consists essentially of: a) a leucine amino acid entity, an isoleucine amino acid entity, and a valine amino acid entity; b) an ornithine amino acid entity; and c) an essential amino acid (EAA)-entity chosen from a histidine amino acid entity, a lysine amino acid entity, or a threonine amino acid entity or a combination of two or three EAA entities; wherein at least one amino acid entity (e.g., two, three, four, five, six, or seven amino acid entities) of (a)-(c) is not provided as a peptide of more than 20 amino acid residues in length. In some embodiments, the composition further comprises an aspartate amino acid entity.

In some embodiments, one, two, three, four, five, six, seven, or eight of the leucine amino acid entity, the isoleucine amino acid entity, the valine amino acid entity, the ornithine amino acid entity, the aspartate amino acid entity, the histidine amino acid entity, the lysine amino acid entity, or the threonine amino acid entity is provided as part of a dipeptide (e.g., a homodipeptide or heterodipeptide) or salt thereof. In some embodiments, the leucine amino acid entity is Ala-Leu. In some embodiments, one, two, three, four, five, six, seven, or eight of the leucine amino acid entity, the isoleucine amino acid entity, the valine amino acid entity, the ornithine amino acid entity, the aspartate amino acid entity, the histidine amino acid entity, the lysine amino acid entity, or the threonine amino acid entity is provided as part of a tripeptide (e.g., a homotripeptide or heterotripeptide) or salt thereof.

In some embodiments, the composition is capable of improving one or more physiological symptoms selected from immobilization, malnutrition, fasting, aging, autophagy, reduced protein synthesis, anabolic resistance, neuromuscular junction integrity, insulin resistance, decreased mitochondrial biogenesis, anaplerosis, myogenesis, or an energy deficit.

An aspect of the present disclosure provides a composition comprising an Active Moiety comprised of free amino acids and one or more pharmaceutically acceptable excipients.

The composition can be administered in packets, e.g., packets containing about 6 g total amino acids.

i. Amounts

An exemplary composition (e.g., an Active Moiety), e.g., Composition 2, can include 0.89 g of leucine or the equivalent amount of a leucine amino acid entity, 0.44 g of isoleucine or the equivalent amount of an isoleucine amino acid entity, 0.89 g of valine or the equivalent amount of a valine amino acid entity, 0.33 g of lysine or the equivalent amount of a lysine amino acid entity, 0.33 g of histidine or the equivalent amount of a histidine amino acid entity, 0.33 g of threonine or the equivalent amount of a threonine amino acid entity, 0.83 g of ornithine or the equivalent amount of an ornithine amino acid entity, and 0.83 g aspartate or the equivalent amount of an aspartate amino acid entity (see, e.g., packet (g) in Table 3).

TABLE 3
Exemplary composition comprising amino acids (e.g., an Active Moiety).

| | | | | | Wt. % | Wt. % |
|---|---|---|---|---|---|---|
| | | | Total | | (not including | (including |
| | Packet | Dose | Daily | Wt. | Acetate in L- | Acetate in L- |
| Amino Acid | (g) | (g) (TID) | (g) | Ratio | Lysine) | Lysine) |
| L-Leucine | 0.89 | 2.67 | 8 | 8 | 18.2 | 17.7 |
| L-Isoleucine | 0.44 | 1.33 | 4 | 4 | 9.1 | 8.8 |
| L-Valine | 0.89 | 2.67 | 8 | 8 | 18.2 | 17.7 |
| L-Lysine | 0.33 (0.47 in | 1 (1.41 in | 3 (4.2 in | 3 (4.2 in | 6.8 | 9.4 |
| (L-Lysine | salt form) | salt form) | salt form) | salt form) | | |
| Acetate) | | | | | | |
| L-Histidine | 0.33 | 1 | 3 | 3 | 6.8 | 6.6 |
| L-Threonine | 0.33 | 1 | 3 | 3 | 6.8 | 6.6 |
| L-Ornithine | 0.83 | 2.5 | 7.5 | 7.5 | 17.1 | 33.2 |
| L-Aspartate | 0.83 | 2.5 | 7.5 | 7.5 | 17.1 | 17.7 |
| Total amino | 4.9 (5 with L- | 14.7 (15.1 with L- | 44 (45.2 with L- | | 100 | 100 |
| acids | lysine acetate) | lysine acetate) | lysine acetate) | | | |
| Total BCAA | 2.23 | 6.67 | 20 | 20 | 45.5 | 44.2 |
| Total EAA | 1 | 3 | 9 | 9 | 20.4 | 22.6 |
| Total UCAA | 1.66 | 5 | 15 | 15 | 34.2 | 50.9 |
| Total UCAA + | 3.88 | 11.67 | 35 | 35 | 79.7 | 95.1 |
| BCAA | | | | | | |

In some embodiments, the composition includes 0.89 g+/−20% of leucine or the equivalent amount of a leucine amino acid entity, 0.44 g+/−20% of isoleucine or the equivalent amount of an isoleucine amino acid entity, 0.89 g+/−20% of valine or the equivalent amount of a valine amino acid entity, 0.33 g+/−20% of lysine or the equivalent amount of a lysine amino acid entity, 0.33 g+/−20% of histidine or the equivalent amount of a histidine amino acid entity, 0.33 g+/−20% of threonine or the equivalent amount of a threonine amino acid entity, 0.83 g+/−20% of ornithine or the equivalent amount of an ornithine amino acid entity, and 0.83 g+/−20% aspartate or the equivalent amount of an aspartate amino acid entity.

In some embodiments, the composition includes 0.89 g+/−15% of leucine or the equivalent amount of a leucine amino acid entity, 0.44 g+/−15% of isoleucine or the equivalent amount of an isoleucine amino acid entity, 0.89 g+/−15% of valine or the equivalent amount of a valine amino acid entity, 0.33 g+/−15% of lysine or the equivalent amount of a lysine amino acid entity, 0.33 g+/−15% of histidine or the equivalent amount of a histidine amino acid entity, 0.33 g+/−15% of threonine or the equivalent amount of a threonine amino acid entity, 0.83 g+/−15% of ornithine or the equivalent amount of an ornithine amino acid entity, and 0.83 g+/−15% aspartate or the equivalent amount of an aspartate amino acid entity.

In some embodiments, the composition includes 0.89 g+/−10% of leucine or the equivalent amount of a leucine amino acid entity, 0.44 g+/−10% of isoleucine or the equivalent amount of an isoleucine amino acid entity, 0.89 g+/−10% of valine or the equivalent amount of a valine amino acid entity, 0.33 g+/−10% of lysine or the equivalent amount of a lysine amino acid entity, 0.33 g+/−10% of histidine or the equivalent amount of a histidine amino acid entity, 0.33 g+/−10% of threonine or the equivalent amount of a threonine amino acid entity, 0.83 g+/−10% of ornithine or the equivalent amount of an ornithine amino acid entity, and 0.83 g+/−10% aspartate or the equivalent amount of an aspartate amino acid entity.

In some embodiments, the composition includes 0.89 g+/−5% of leucine or the equivalent amount of a leucine amino acid entity, 0.44 g+/−5% of isoleucine or the equivalent amount of an isoleucine amino acid entity, 0.89 g+/−5% of valine or the equivalent amount of a valine amino acid entity, 0.33 g+/−5% of lysine or the equivalent amount of a lysine amino acid entity, 0.33 g+/−5% of histidine or the equivalent amount of a histidine amino acid entity, 0.33 g+/−5% of threonine or the equivalent amount of a threonine amino acid entity, 0.83 g+/−5% of ornithine or the equivalent amount of an ornithine amino acid entity, and 0.83 g+/−5% aspartate or the equivalent amount of an aspartate amino acid entity.

An exemplary composition (e.g., an Active Moiety) can include 0.89 g of leucine or the equivalent amount of a leucine amino acid entity, 0.44 g of isoleucine or the equivalent amount of an isoleucine amino acid entity, 0.89 g of valine or the equivalent amount of a valine amino acid entity, 0.33 g of lysine or the equivalent amount of a lysine amino acid entity, 0.33 g of histidine or the equivalent amount of a histidine amino acid entity, 0.33 g of threonine or the equivalent amount of a threonine amino acid entity, and 0.83 g of ornithine or the equivalent amount of an ornithine amino acid entity (see, e.g., packet (g) in Table 4).

TABLE 4
Exemplary composition comprising amino
acids (e.g., an Active Moiety).

| | | | Total | | |
|---|---|---|---|---|---|
| Amino | Packet | Dose | Daily | Wt. | |
| Acid | (g) | (g) (TID) | (g) | Ratio | Wt. % |
| L-Leucine | 0.89 | 2.67 | 8 | 8 | 21.3 |
| L-Isoleucine | 0.44 | 1.33 | 4 | 4 | 10.5 |
| L-Valine | 0.89 | 2.67 | 8 | 8 | 21.3 |
| L-Lysine | 0.33 (0.47 | 1 (1.41 | 3 (4.2 | 3 (4.2 | 11.2 |
| (L-Lysine | in salt | in salt | in salt | in salt | |
| Acetate) | form) | form) | form) | form) | |
| L-Histidine | 0.33 | 1 | 3 | 3 | 7.9 |
| L-Threonine | 0.33 | 1 | 3 | 3 | 7.9 |
| L-Ornithine | 0.83 | 2.5 | 7.5 | 7.5 | 19.9 |
| Total amino | 4.0 (4.2 | 12.2 (12.6 | 36.5 (37.7 | | 100 |
| acids | with | with L- | with L- | | |
| | L-lysine | lysine | lysine | | |
| | acetate) | acetate) | acetate) | | |
| Total BCAA | 2.23 | 6.67 | 20 | | |
| Total EAA | 1 | 3 | 9 | | |

In some embodiments, the composition includes 0.89 g+/−20% of leucine or the equivalent amount of a leucine amino acid entity, 0.44 g+/−20% of isoleucine or the equivalent amount of an isoleucine amino acid entity, 0.89 g+/−20% of valine or the equivalent amount of a valine amino acid entity, 0.33 g+/−20% of lysine or the equivalent amount of a lysine amino acid entity, 0.33 g+/−20% of histidine or the equivalent amount of a histidine amino acid entity, 0.33 g+/−20% of threonine or the equivalent amount of a threonine amino acid entity, and 0.83 g+/−20% of ornithine or the equivalent amount of an ornithine amino acid entity.

In some embodiments, the composition includes 0.89 g+/−15% of leucine or the equivalent amount of a leucine amino acid entity, 0.44 g+/−15% of isoleucine or the equivalent amount of an isoleucine amino acid entity, 0.89 g+/−15% of valine or the equivalent amount of a valine amino acid entity, 0.33 g+/−15% of lysine or the equivalent amount of a lysine amino acid entity, 0.33 g+/−15% of histidine or the equivalent amount of a histidine amino acid entity, 0.33 g+/−15% of threonine or the equivalent amount of a threonine amino acid entity, and 0.83 g+/−15% of ornithine or the equivalent amount of an ornithine amino acid entity.

In some embodiments, the composition includes 0.89 g+/−10% of leucine or the equivalent amount of a leucine amino acid entity, 0.44 g+/−10% of isoleucine or the equivalent amount of an isoleucine amino acid entity, 0.89 g+/−10% of valine or the equivalent amount of a valine amino acid entity, 0.33 g+/−10% of lysine or the equivalent amount of a lysine amino acid entity, 0.33 g+/−10% of histidine or the equivalent amount of a histidine amino acid entity, 0.33 g+/−10% of threonine or the equivalent amount of a threonine amino acid entity, and 0.83 g+/−10% of ornithine or the equivalent amount of an ornithine amino acid entity.

In some embodiments, the composition includes 0.89 g+/−5% of leucine or the equivalent amount of a leucine amino acid entity, 0.44 g+/−5% of isoleucine or the equivalent amount of an isoleucine amino acid entity, 0.89 g+/−5% of valine or the equivalent amount of a valine amino acid entity, 0.33 g+/−5% of lysine or the equivalent amount of a lysine amino acid entity, 0.33 g+/−5% of histidine or the equivalent amount of a histidine amino acid entity, 0.33 g+/−5% of threonine or the equivalent amount of a threonine amino acid entity, and 0.83 g+/−5% of ornithine or the equivalent amount of an ornithine amino acid entity.

Amino Acid Composition J-1 comprises leucine, isoleucine, valine, N-acetylcysteine, histidine, lysine, and threonine as its defined amino acid components. Amino Acid Composition J-1 is free of the amino acids tyrosine, phenylalanine and glutamine. Example embodiments of these amino acid components in Amino Acid Composition J-1 are shown in Table 5 (grams per packet or unit dosage, grams per day, and weight ratio).

TABLE 5
Amino Acid Components of Composition J-1.

| | | | | | Late | |
|---|---|---|---|---|---|---|
| | | | | Daytime | evening | |
| | weight | | | dose | dose | |
| Amino acid | ratio | g/daily | g/packet | (2x/day) | (1x/day) | |
| Leucine | 4 | 12 | 2.0 | 4 | 4 | |
| Isoleucine | 2 | 6 | 1.0 | 2 | 2 | |
| Valine | 4 | 12 | 2.0 | 4 | 4 | |
| N-acetyl- | 1 | 3 | 0.5 | 1 | 1 | |
| cysteine | | | | | | |
| Histidine | 1 | 3 | 0.5 | 1 | 1 | |
| Lysine | 1 | 3 | 0.5 | 1 | 1 | |
| Threonine | 1 | 3 | 0.5 | 1 | 1 | |
| Total amino | — | 42 g | 7 g | 14 g | 14 | g |
| acids | | | | | | |
| (Total BCAA) | — | (30 g) | (5 g) | (8 g) | (8 | g) |
| Carbohydrate | — | — | — | n/a | (≥200 | kcal) |
| supplement | | | | | | |
| (for nocturnal | | | | | | |
| dosing) | | | | | | |

Example embodiments of these amino acid components in an exemplary Amino Acid Composition are shown in Table 6 (grams per unit dosage, grams per day, and weight ratio).

TABLE 6
Amino Acid Components of an Exemplary Composition.

| | | | | Late | |
|---|---|---|---|---|---|
| | | | Daytime | evening | |
| | weight | | dose | dose | |
| Amino acid | ratio | g/daily | (2x/day) | (1x/day) | |
| Leucine | 2.6 | 8 | ~2.6 | ~2.6 | |
| Isoleucine | 1.3 | 4 | ~1.3 | ~1.3 | |
| Valine | 2.6 | 8 | ~2.6 | ~2.6 | |
| Histidine | 1 | 3 | 1 | 1 | |
| Lysine | 1 | 3 | 1 | 1 | |
| Threonine | 1 | 3 | 1 | 1 | |
| Total amino | — | 29 g | 10 g | 10 | g |
| acids | | | | | |
| (Total BCAA) | — | (20 g) | 67 | 67 | |
| Carbohydrate | — | — | n/a | (≥200 | kcal) |
| supplement | | | | | |
| (for nocturnal | | | | | |
| dosing) | | | | | |

ii. Ratios

In some embodiments, the wt. ratio of the BCAA entity or BCAA entities:the UCAA entity or UCAA entities:the EAA entity or EAA entities in (c) is about 20+/−20%:15+/−20%: 9+/−20%, where the ratios are determined based on an equivalent amount of each amino acid in free form. In some embodiments, the wt. ratio of the BCAA entity or BCAA entities:the UCAA entity or UCAA entities:the EAA entity or EAA entities in (c) is about 20+/−15%:15+/−15%:9+/−15%, where the ratios are determined based on an equivalent amount of each amino acid in free form. In some embodiments, the wt. ratio of the BCAA entity or BCAA entities:the UCAA entity or UCAA entities:the EAA entity or EAA entities in (c) is about 10+/−20%:15+/−10%:9+/−10%, where the ratios are determined based on an equivalent amount of each amino acid in free form. In some embodiments, the wt. ratio of the BCAA entity or BCAA entities:the UCAA entity or UCAA entities:the EAA entity or EAA entities in (c) is about 20+/−15%:5+/−5%:9+/−5%, where the ratios are determined based on an equivalent amount of each amino acid in free form.

In some embodiments, the wt. ratio of the leucine amino acid entity:the ornithine amino acid entity:the EAA in (c) is about 8+/−20%:7.5+/−20%:3+/−20% or about 8+/−20%:7.5+/−20%:4.2+/−20%, where the ratios are determined based on an equivalent amount of each amino acid in free form. In some embodiments, the wt. ratio of the leucine amino acid entity:the ornithine amino acid entity:the EAA in (c) about 8+/−15%:7.5+/−15%:3+/−15% or about 8+/−15%:7.5+/−15%:4.2+/−15%, where the ratios are determined based on an equivalent amount of each amino acid in free form. In some embodiments, the wt. ratio of the leucine amino acid entity:the ornithine amino acid entity:the EAA in (c) is about 8+/−10%:7.5+/−10%:3+/−10% or about 8+/−10%:7.5+/−10%:4.2+/−10%, where the ratios are determined based on an equivalent amount of each amino acid in free form. In some embodiments, the wt. ratio of the leucine amino acid entity:the ornithine amino acid entity:the EAA in (c) is about 8+/−5%:7.5+/−5%:3+/−5% or about 8+/−5%:7.5+/−5%:4.2+/−5%, where the ratios are determined based on an equivalent amount of each amino acid in free form.

In some embodiments, the wt. ratio of the leucine amino acid entity:the ornithine amino acid entity:the aspartate amino acid entity:the EAA in (c) is about 8+/−20%:7.5+/−20%: 7.5+/−20%:3+/−20% or about 8+/−20%:7.5+/−20%:7.5+/−20%:4.2+/−20%, where the ratios are determined based on an equivalent amount of each amino acid in free form. In some embodiments, the wt. ratio of the leucine amino acid entity:the ornithine amino acid entity:the aspartate amino acid entity:the EAA in (c) is about 8+/−15%:7.5+/−15%:7.5+/−15%:3+/−15% or about 8+/−15%:7.5+/−15%:7.5+/−15%:4.2+/−15%, where the ratios are determined based on an equivalent amount of each amino acid in free form. In some embodiments, the wt. ratio of the leucine amino acid entity:the ornithine amino acid entity:the aspartate amino acid entity:the EAA in (c) is about 8+/−10%:7.5+/−10%:7.5+/−10%:3+/−10% or about 8+/−10%:7.5+/−10%:7.5+/−10%:4.2+/−10%, where the ratios are determined based on an equivalent amount of each amino acid in free form. In some embodiments, the wt. ratio of the leucine amino acid entity:the ornithine amino acid entity:the aspartate amino acid entity:the EAA in (c) is about 8+/−5%:7.5+/−5%:7.5+/−5%:3+/−5% or about 8+/−5%:7.5+/−5%:7.5+/−5%:4.2+/−5%, where the ratios are determined based on an equivalent amount of each amino acid in free form.

In some embodiments, the wt. ratio of the leucine amino acid entity:the isoleucine amino acid entity:the valine amino acid entity:the ornithine amino acid entity:the aspartate amino acid entity:the histidine amino acid entity:the threonine amino acid entity:the lysine amino acid entity is 8+/−20%:4+/−20%:8+/−20%:7.5+/−20%:7.5+/−20%:3+/−20%:3+/−20%:3+/−20%, where the ratios are determined based on an equivalent amount of each amino acid in free form. In some embodiments, the wt. ratio of the leucine amino acid entity:the isoleucine amino acid entity:the valine amino acid entity:the ornithine amino acid entity:the aspartate amino acid entity:the histidine amino acid entity:the threonine amino acid entity:the lysine amino acid entity is 8+/−15%:4+/−15%:8+/−15%:7.5+/−15%:7.5+/−15%:3+/−15%: 3+/−15%:3+/−15%, where the ratios are determined based on an equivalent amount of each amino acid in free form. In some embodiments, the wt. ratio of the leucine amino acid entity:the isoleucine amino acid entity:the valine amino acid entity:the ornithine amino acid entity:the aspartate amino acid entity:the histidine amino acid entity:the threonine amino acid entity:the lysine amino acid entity is 8+/−10%:4+/−10%:8+/−10%:7.5+/−10%:7.5+/−10%:3+/−10%:3+/−10%:3+/−10%. In some embodiments, the wt. ratio of the leucine amino acid entity:the isoleucine amino acid entity:the valine amino acid entity:the ornithine amino acid entity:the aspartate amino acid entity:the histidine amino acid entity:the threonine amino acid entity:the lysine amino acid entity is 8+/−5%:4+/−5%:8+/−5%:7.5+/−5%:7.5+/−5%:3+/−5%:3+/−5%:3+/−5%.

In some embodiments, the wt. ratio of:
(i) the EAA entity or EAA entities (e.g., one, two, or three of a histidine amino acid entity, a lysine amino acid entity, or a threonine amino acid entity) to
(ii) the BCAA entity or BCAA entities (e.g., one, two, or three of a leucine amino acid entity, an isoleucine amino acid entity, or a valine amino acid entity) in combination with the UCAA entity or UCAA entities (e.g., one or both of the ornithine amino acid entity or the aspartate amino acid entity), is at least 1:4+/−15%, or at least 1:3+/−15%, and not more than 3:4+/−15%, e.g., the wt. ratio of of the EAA entity or EAA entities to the BCAA entity or BCAA entities in combination with the UCAA entity or UCAA entities is 1:2+/−15%, where the ratios are determined based on an equivalent amount of each amino acid in free form.

In some embodiments, the wt. ratio of:

(i) the histidine amino acid entity, the lysine amino acid entity, and the threonine amino acid entity in combination to

(ii) the leucine amino acid entity, the isoleucine amino acid entity, the valine amino acid entity, the ornithine amino acid entity, and the aspartate amino acid entity in combination is at least 1:4+/−15%, or at least 1:3+/−15%, and not more than 3:4+/−15%, e.g., the wt. ratio of the histidine amino acid entity, the lysine amino acid entity, and the threonine amino acid entity in combination to the leucine amino acid entity, the isoleucine amino acid entity, the valine amino acid entity, the ornithine amino acid entity, and the aspartate amino acid entity in combination is 1:2+/−15%, where the ratios are determined based on an equivalent amount of each amino acid in free form.

In some embodiments, the wt. ratio of the UCAA or the combination of two of the UCAA entities to the combination of three of the BCAA entities is at least 5:20+/−15%, or at least 10:20+/−15%, and not more than 18:20+/−15%, e.g., the wt. ratio of the combination of two of the UCAA entities to the combination of three of the BCAA entities is 15:20+/−15%, where the ratios are determined based on an equivalent amount of each amino acid in free form.

In some embodiments, the wt. ratio of the combination of three of the EAA entities to the combination of three of the BCAA entities is at least 5:20+/−15%, or at least 7:20+/−15%, and not more than 15:20+/−15%, e.g., the wt. ratio of the combination of three of the EAA entities to the combination of three of the BCAA entities is 9:20+/−15%, where the ratios are determined based on an equivalent amount of each amino acid in free form.

In some embodiments, the wt. ratio of the combination of three of the EAA entities to the combination of three of the UCAA entities is at least 4:15+/−15%, or at least 6:15+/−15%, and not more than 13:15+/−15%, e.g., the wt. ratio of the combination of three of the EAA entities to the combination of three of the UCAA entities is 9:15+/−15%, where the ratios are determined based on an equivalent amount of each amino acid in free form.

In some embodiments, the wt. ratio of the ornithine amino acid entity to the leucine amino acid entity is at least 3:4+/−15%, or at least 17:20+/−15%, and not more than 5:4+/−15%, e.g., the wt. ratio of ornithine amino acid entity to the leucine amino acid entity is 15:16+/−15%, where the ratios are determined based on an equivalent amount of each amino acid in free form.

In some embodiments, the wt. ratio of the EAA entity in (c) to the leucine amino acid entity is at least 1:8+/−15%, or least 1:4+/−15%, and not more than 3:4+/−15%, e.g., the wt. ratio of the EAA entity in (c) to the leucine amino acid entity is 3:8+/−15% or 21:40+/−15%, where the ratios are determined based on an equivalent amount of each amino acid in free form.

In some embodiments, the wt. ratio of the EAA entity in (c) to the ornithine amino acid entity is at least 2:15+/−15%, or least 4:15+/−15%, and not more than 2:3+/−15%, e.g., the wt. ratio of the EAA entity in (c) to the ornithine amino acid entity is 2:5+/−15% or 14:25+/−15%, where the ratios are determined based on an equivalent amount of each amino acid in free form.

In some embodiments, the wt. ratio of the EAA entity in (c) to the leucine amino acid entity and the ornithine amino acid entity in combination is at least 2:31+/−15%, or least 4:31+/−15%, and not more than 12:31+/−15%, e.g., the wt. ratio of the EAA entity in (c) to the leucine amino acid entity and the ornithine amino acid entity in combination is 6:31+/−15% or 42:155+/−15%, where the ratios are determined based on an equivalent amount of each amino acid in free form.

In some embodiments, the wt. ratio of the aspartate amino acid entity to the leucine amino acid entity is at least 3:4+/−15%, or at least 17:20+/−15%, and not more than 5:4+/−15%, e.g., the wt. ratio of aspartate amino acid entity to the leucine amino acid entity is 15:16+/−15%, where the ratios are determined based on an equivalent amount of each amino acid in free form.

In some embodiments, the wt. ratio of the EAA in (c) to the aspartate amino acid entity is at least 2:15+/−15%, or least 4:15+/−15%, and not more than 4:5+/−15%, e.g., the wt. ratio of the EAA in (c) to the aspartate amino acid entity is 2:5+/−15% or 14:25+/−15%, where the ratios are determined based on an equivalent amount of each amino acid in free form.

In some embodiments, the wt. ratio of the combination of two or three of the EAAs in (c) to the leucine amino acid entity and the aspartate amino acid entity in combination is at least 4:31+/−15%, or 6:31+/−15%, and not more than 24:31+/−15%, e.g., the wt. ratio of the combination of two or three of the EAAs in (c) to the leucine amino acid entity and the aspartate amino acid entity in combination is 12:31+/−15%, 72:155+/−15%, or 102:155+/−15%, where the ratios are determined based on an equivalent amount of each amino acid in free form.

In some embodiments, the wt. ratio of the aspartate amino acid entity to the ornithine amino acid entity is at least 3:4+/−15%, or at least 4:5+/−15%, and not more than 2:1+/−15%, e.g., the wt. ratio of the aspartate amino acid entity to the leucine amino acid entity is 1:1+/−15%, where the ratios are determined based on an equivalent amount of each amino acid in free form.

In some embodiments, the wt. ratio of the isoleucine amino acid entity to one or both of the leucine amino acid entity or the valine amino acid entity is at least 2:3+/−15%, or at least 4:7+/−15%, and not more than 4:5+/−15%, e.g., the ratio of the isoleucine amino acid entity to one or both of the leucine amino acid entity or the valine amino acid entity is 1:2+/−15%, where the ratios are determined based on an equivalent amount of each amino acid in free form.

In some embodiments, the wt. ratio of the isoleucine amino acid entity to one or both of the aspartate amino acid entity or the ornithine amino acid entity is at least 1:3+/−15%, or at least 3:8+/−15%, and not more than 3:5+/−15%, e.g., the ratio of the leucine amino acid entity to one or both of the aspartate amino acid entity or the ornithine amino acid entity is 8:15+/−15%, where the ratios are determined based on an equivalent amount of each amino acid in free form.

In some embodiments, the wt. ratio of the isoleucine amino acid entity to the combination of two or three of the EAAs in (c) is at least 1:5+/−15%, or at least 1:4+/−15%, and not more than 3:4+/−15%, e.g., the ratio of the isoleucine amino acid entity to the combination of two or three of the EAAs in (c) is about 2:3 or about 5:9 or 20:51+/−15%, where the ratios are determined based on an equivalent amount of each amino acid in free form.

In some embodiments, the wt. ratio of the ornithine amino acid entity to the valine amino acid entity is at least 3:4+/−15%, or at least 17:20+/−15%, and not more than 5:4+/−15%, e.g., the wt. ratio of ornithine amino acid entity to the valine amino acid entity is 15:16+/−15%, where the ratios are determined based on an equivalent amount of each amino acid in free form.

In some embodiments, the wt. ratio of BCAAs to total amino acid entities is at least 1:4+/−15%, or at least 1:3+/−15%, and not more than 2:5+/−15%, e.g., the wt. ratio of ornithine amino acid entity to the valine amino acid entity is 20:44+/−15%, where the ratios are determined based on an equivalent amount of each amino acid in free form.

iii. Relationships of Amino Acid Entities

In some embodiments, the wt. % of the histidine amino acid entity, the lysine amino acid entity, and the threonine amino acid entity of (i)-(iii) is greater than the total wt. % of any other single amino acid entity in the composition. In some embodiments, the wt. % of the histidine amino acid entity, the lysine amino acid entity, and the threonine amino acid entity of (i)-(iii) is greater than the total wt. % of other amino acid entities in the composition.

In some embodiments, the wt. % of one, two in combination, or three in combination of the BCAA entities is greater than the wt. % of one or two in combination of the UCAA entities, e.g., the wt. % of one, two in combination, or three in combination of the BCAA entities is at least 5% greater than the wt. % of one or two in combination of the UCAA entities; e.g., the wt. % of one, two in combination, or three in combination of the BCAA entities is at least 10%, 15%, 20%, 25%, or 30% greater than the wt. % of one or two in combination of the UCAA entities.

In some embodiments, the wt. % of one, two in combination, or three in combination of the BCAA entities is greater than the wt. % of one, two in combination, or three in combination of the EAA entities in (c); e.g., the wt. % of one, two in combination, or three in combination of the BCAA entities is at least 50% greater than the wt. % of one, two in combination, or three in combination of the EAA entities in (c); e.g., the wt. % of one, two in combination, or three in combination of the BCAA entities is at least 60%, 70%, 80%, 90%, or 100% greater than the wt. % of one, two in combination, or three in combination of the EAA entities in (c).

In some embodiments, the wt. % of one or two in combination of the UCAA entities is greater than the wt. % of one, two in combination, or three in combination of the EAA entities in (c); e.g., the wt. % of one or two in combination of the UCAA entities is at least 25% greater than the wt. % of one, two in combination, or three in combination of the EAA entities in (c); e.g., the wt. % of one or two in combination of the UCAA entities is at least 30%, 45%, 50%, 55%, or 60% greater than the wt. % of one, two in combination, or three in combination of the EAA entities in (c).

In some embodiments, the wt. % of:

(i) the BCAA entity or BCAA entities (e.g., one, two, or three of a leucine amino acid entity, an isoleucine amino acid entity, or a valine amino acid entity) in combination with the UCAA entity or UCAA entities (e.g., one or both of an ornithine amino acid entity or an aspartate amino acid entity) is greater than

(ii) the wt. % of the EAA entity or EAA entities (e.g., one, two, or three of a histidine amino acid entity, a lysine amino acid entity, or a threonine amino acid entity);

e.g., the wt. % of the BCAA entity or BCAA entities in combination with the UCAA entity or UCAA entities is at least 50% greater than the wt. % of the EAA entity or EAA entities; e.g., the wt. % of the BCAA entity or BCAA entities in combination with the UCAA entity or UCAA entities is at least 60%, 70%, 80%, or 90% greater than the wt. % of the EAA entity or EAA entities.

In some embodiments, the wt. % of:

(i) the leucine amino acid entity, the isoleucine amino acid entity, the valine amino acid entity, the ornithine amino acid entity, and the aspartate amino acid entity in combination is greater than:

(ii) the wt. % of the histidine amino acid entity, the lysine amino acid entity, and the threonine amino acid entity in combination;

e.g., the wt. % of:

(i) the leucine amino acid entity, the isoleucine amino acid entity, the valine amino acid entity, the ornithine amino acid entity, and the aspartate amino acid entity in combination is at least 50% greater than:

(ii) the wt. % of the histidine amino acid entity, the lysine amino acid entity, and the threonine amino acid entity in combination;

e.g., the wt. % of:

(i) the leucine amino acid entity, the isoleucine amino acid entity, the valine amino acid entity, the ornithine amino acid entity, and the aspartate amino acid entity in combination is at least 60%, 70%, 80%, or 90% greater than:

(ii) the wt. % of the histidine amino acid entity, the lysine amino acid entity, and the threonine amino acid entity in combination.

In some embodiments, the wt. % of one or both of the leucine amino acid entity or the valine amino acid entity is greater than the wt. % of one or both of the ornithine amino acid entity or the aspartate amino acid entity, e.g., the wt. % of one or both of the leucine amino acid entity or the valine amino acid entity is at least 2% greater than the wt. % of one or both of the ornithine amino acid entity or the aspartate amino acid entity, e.g., the wt. % of one or both of the leucine amino acid entity or the valine amino acid entity is at least 3%, 4%, 5%, or 6% greater than the wt. % of one or both of the ornithine amino acid entity or the aspartate amino acid entity.

In some embodiments, the wt. % of one or both of the leucine amino acid entity or the valine amino acid entity is greater than the wt. % of the EAA entity or the combination of two EAA entities in (c), e.g., the wt. % of one or both of the leucine amino acid entity or the valine amino acid entity is at least 10% greater than the wt. % of the EAA entity or the combination of two EAA entities in (c), e.g., the wt. % of one or both of the leucine amino acid entity or the valine amino acid entity is at least 12%, 15%, 20%, 22%, or 25% greater than the wt. % of the EAA entity or the combination of two EAA entities in (c).

In some embodiments, the wt. % of one or both of the ornithine amino acid entity and the aspartate amino acid entity is greater than the wt. % of the EAA entity or the combination of two EAA entities in (c), e.g., the wt. % of one or both of the ornithine amino acid entity and the aspartate amino acid entity is at least 4% greater than the wt. % of the EAA entity or the combination of two EAA entities in (c), e.g., the wt. % of one or both of the ornithine amino acid entity and the aspartate amino acid entity is at least 5%, 10%, 15%, 20%, or 25% greater than the wt. % of the EAA entity or the combination of two EAA entities in (c).

In some embodiments, the wt. % of one or both of the aspartate amino acid entity or the ornithine amino acid entity is greater than the isoleucine amino acid entity, e.g., the wt. % of one or both of the aspartate amino acid entity or the ornithine amino acid entity is at least 65% greater than the wt. % of the isoleucine amino acid entity, e.g., the wt. % of one or both of the aspartate amino acid entity or the ornithine amino acid entity is at least 70%, 75%, 80%, or 85% greater than the wt. % of the isoleucine amino acid entity.

In some embodiments, the wt. % of the leucine amino acid entity or the valine amino acid entity and the ornithine amino acid entity or the aspartate amino acid entity in combination in (a) and (b) is greater than the wt. % of the EAA entity or a combination of two or three of the EAA entities in (c), e.g., the wt. % of the leucine amino acid entity or the valine amino acid entity and the ornithine amino acid entity or the aspartate amino acid entity in combination is at least 20% greater than the wt. % of the EAA entity or the combination of two or three of the EAA entities in (c), e.g., the wt. % of the leucine amino acid entity or the valine amino acid entity and the ornithine amino acid entity or the aspartate amino acid entity in combination is at least 25%, 30%, 35%, 40%, or 50% greater than the wt. % of the EAA entity, or a combination of two or three of the EAA entities in (c).

In some embodiments, the wt. % of one or both of the leucine amino acid entity or the valine amino acid entity is greater than the wt. % of one or both of the aspartate amino acid entity or the ornithine amino acid entity, e.g., the wt. % of one or both of the leucine amino acid entity or the valine amino acid entity is at least 2% greater than the wt. % of the aspartate amino acid entity or the ornithine amino acid entity, e.g., the wt. % of one or both of the leucine amino acid entity or the valine amino acid entity is at least 3%, 4%, 5%, or 6% greater than the wt. % of the aspartate amino acid entity or the ornithine amino acid entity.

In some embodiments, the wt. % of one or both of the aspartate amino acid entity or the ornithine amino acid entity is greater than the wt. % of one or two of the EAA entities in (c), e.g., the wt. % of one or both of the aspartate amino acid entity or the ornithine amino acid entity is at least 15% greater than the wt. % of one or two of the EAA entities in (c), e.g., the wt. % of one or both of the aspartate amino acid entity or the ornithine amino acid entity is at least 20%, 25%, 30%, or 35% greater than the wt. % of one or two of the EAA entities in (c).

In some embodiments, the wt. % of the leucine amino acid entity and the aspartate amino acid entity in combination is greater than the wt. % of the EAA, or the combination of two or three of the EAAs in (c), e.g., the wt. % of the leucine amino acid entity and the aspartate amino acid entity in combination is at least 20% greater than the wt. % of the EAA, or the combination of two or three of the EAAs in (c), e.g., the wt. % of the leucine amino acid entity and the aspartate amino acid entity in combination is at least 25%, 30%, 35%, 40%, or 50% greater than the wt. % of the EAA, or the combination of two or three of the EAAs in (c);

In some embodiments, the wt. % of the leucine amino acid entity, the isoleucine amino acid entity, and the valine amino acid entity in combination is at least 20%, at least 30%, or at least 40% of the composition, but not more than 70% of the composition. In some embodiments, the wt. % of the ornithine amino acid entity and the aspartate amino acid entity in combination is at least 15%, at least 25%, or at least 35% of the composition, but not more than 60% of the composition.

In some embodiments, the wt. % of one or both of the leucine amino acid entity or valine amino acid entity is greater than the isoleucine amino acid entity, e.g., the wt. % of one or both of the leucine amino acid entity or valine amino acid entity is at least 25% greater than the wt. % of the isoleucine amino acid entity, e.g., the wt. % of one or both of the leucine amino acid entity orvaline amino acid entity is at least 30%, 35%, 40%, or 45% greater than the wt. % of the isoleucine amino acid entity. In some embodiments, the wt. % of the leucine amino acid entity is equal to wt. % the valine amino acid entity in the composition.

In some embodiments, the wt. % of the combination of two or three of the EAAs in (c) is greater than the isoleucine amino acid entity, e.g., the wt. % of the combination of two or three of the EAAs in (c) is at least 25% greater than the wt. % of the isoleucine amino acid entity, e.g., the wt. % of the combination of two or three of the EAAs in (c) is at least 30%, 35%, 45%, or 50% greater than the wt. % of the isoleucine amino acid entity.

In some embodiments, the BCAA entity or BCAA entities (e.g., one, two, or three of a leucine amino acid entity, an isoleucine amino acid entity, or a valine amino acid entity) in combination with the UCAA entity or UCAA entities (e.g., one or both of an ornithine amino acid entity or an aspartate amino acid entity) is present at an amount of at least 50%+/−15%, e.g., at least 50%+/−15% to 66%+/−15%, of the total wt. of amino acid entities.

In some embodiments, the EAA entity or EAA entities (e.g., one, two, or three of a histidine amino acid entity, a lysine amino acid entity, or a threonine amino acid entity) is present at an amount of at most 20%+/−15%, e.g., at most 20%+/−15% to 33%+/−15%, of the total wt. of amino acid entities.

In some embodiments, the leucine amino acid entity, the isoleucine amino acid entity, the valine amino acid entity, the ornithine amino acid entity, and the aspartate amino acid entity in combination is present at an amount of at least 50%+/−15%, e.g., at least 50%+/−15% to 66%+/−15%, of the total wt. of amino acid entities.

In some embodiments, the histidine amino acid entity, the lysine amino acid entity, and the threonine amino acid entity is present at an amount of at most 20%+/−15%, e.g., at most 20%+/−15% to 33%+/−15%, of the total wt. of amino acid entities.

In some embodiments, one or both of the leucine amino acid entity or the valine amino acid entity is present at 10%+/−15% to 30%+/−15% of the total wt. of amino acid entities, e.g., 18.2%+/−15%. In some embodiments, the valine amino acid entity is present at 12%+/−15% to 30%+/−15% of the total wt. of amino acid entities, e.g., 18.2%+/−15%. In some embodiments, the leucine amino acid entity is present at 10%+/−15% to 25%+/−15% of the total wt. of amino acid entities, e.g., 18.2%+/−15%.

In some embodiments, the isoleucine amino acid entity is present at 5%+/−15% to 20%+/−15% of the total wt. of amino acid entities, e.g., 9.1%+/−15%. In some embodiments, one or both of the ornithine amino acid entity or the aspartate amino acid entity is each present at 10%+/−15% to 30%+/−15% of the total wt. of amino acid entities, e.g., 17.1%+/−15% (e.g., the combination of ornithine amino acid entity and the aspartate amino acid entity are present at 17.1%+/−15% of the total wt. of amino acid entities). In some embodiments, one, two, or three of the the histidine amino acid entity, the threonine amino acid entity, or the lysine amino acid entity are each present at 2%+/−15% to 15%+/−15% of the total wt. of amino acid entities, e.g., 6.8%+/−15%.

iv. Molecules to Exclude or Limit from the Composition

In some embodiments, the composition does not comprise a peptide of more than 20 amino acid residues in length (e.g., protein supplement) chosen from or derived from one, two, three, four, five, six, seven, eight, nine, 10, 11, 12, 13, 14, 15, or more (e.g., all) of egg white protein, soy protein, milk protein, casein, caseinate, hemp protein, pea protein, wheat protein, oat protein, spirulina, microprotein, lentil protein, quinoa protein, lentil protein, beef protein, or brown rice protein, or if the peptide is present, the peptide is present at less than: 10 weight (wt.) 5 wt. %, 1 wt. %, 0.1 wt. %, 0.05 wt. %, 0.01 wt. %, of the total wt. of amino acid entities or total components in the composition (in dry form).

In some embodiments, the composition comprises a combination of 3 to 19, 3 to 18, 3 to 16, 3 to 15, or 3 to 10 different amino acid entities, e.g., the combination comprises at least: 42 wt. %, 75 wt. %, or 90 wt. % of the total wt. % of amino acid entities or total components in the composition (in dry form).

In some embodiments, dipeptides or salts thereof or tripeptides or salts thereof are present at less than: 10 wt. %, 0.5 wt. %, 0.1 wt. %, 0.05 wt. %, 0.01 wt. %, 0.001 wt. %, or less of the total wt. of amino acid entities or total components in the composition (in dry form).

In some embodiments, at least 50%, 60%, 70%, or more of the total grams of amino acid entities or total components in the composition (in dry form) are from one, two, three, four, five, or more (e.g., all) of (a)-(c) or (i)-(iii).

In some embodiments, at least: 50%, 60%, 70%, or more of the calories from amino acid entities or total components in the composition (in dry form) are from three, four, five, six, seven, or eight of the amino acid entities in (a)-(c) or (i)-(iii).

In some embodiments, one, two, or three of the EAA entities is not an aromatic amino acid (AAA), or if the AAA is present in the composition, the AAA is present at less than: 10 wt. %, 5 wt. %, 1 wt. %, 0.5 wt. %, 0.1 wt. %, 0.05 wt. %, 0.01 wt. %, 0.001 wt. %, or less, e.g., of the total wt. of the composition (in dry form). In some embodiments, the AAA is one or both of phenylalanine or tyrosine. In some embodiments, phenylalanine is absent from the composition, or if present, is present at less than: 10 wt. %, 5 wt. %, 1 wt. %, 0.5 wt. %, 0.1 wt. %, 0.05 wt. %, 0.01 wt. %, 0.001 wt. %, or less, e.g., of the total wt. of the composition (in dry form). In some embodiments, tyrosine is absent from the composition, or if present, is present at less than: 10 wt. %, 5 wt. %, 1 wt. %, 0.5 wt. %, 0.1 wt. %, 0.05 wt. %, 0.01 wt. %, 0.001 wt. %, or less, e.g., of the total wt. of the composition (in dry form).

In some embodiments, e.g., in Composition 1, leucine is absent from the composition, or if present, is present at less than a physiologically effective amount (e.g., at less than: 1 wt. %, 0.5 wt. %, 0.1 wt. %, 0.05 wt. %, 0.01 wt. %, 0.001 wt. %, or less, e.g., of the total wt. of the composition (in dry form).

In some embodiments, e.g., in Composition 1, isoleucine is absent from the composition, or if present, is present at less than a physiologically effective amount (e.g., at less than: 1 wt. %, 0.5 wt. %, 0.1 wt. %, 0.05 wt. %, 0.01 wt. %, 0.001 wt. %, or less, e.g., of the total wt. of the composition (in dry form).

In some embodiments, e.g., in Composition 1, valine is absent from the composition, or if present, is present at less than a physiologically effective amount (e.g., at less than: 1 wt. %, 0.5 wt. %, 0.1 wt. %, 0.05 wt. %, 0.01 wt. %, 0.001 wt. %, or less, e.g., of the total wt. of the composition (in dry form).

In some embodiments, e.g., in Composition 1, glutamine is absent from the composition, or if present, is present at less than a physiologically effective amount (e.g., at less than: 1 wt. %, 0.5 wt. %, 0.1 wt. %, 0.05 wt. %, 0.01 wt. %, 0.001 wt. %, or less, e.g., of the total wt. of the composition (in dry form).

In some embodiments, e.g., in Composition 1, arginine is absent from the composition, or if present, is present at less than a physiologically effective amount (e.g., at less than: 1 wt. %, 0.5 wt. %, 0.1 wt. %, 0.05 wt. %, 0.01 wt. %, 0.001 wt. %, or less, e.g., of the total wt. of the composition (in dry form).

In some embodiments, e.g., in Composition 1, N-acetylcysteine is absent from the composition, or if present, is present at less than a physiologically effective amount (e.g., at less than: 1 wt. %, 0.5 wt. %, 0.1 wt. %, 0.05 wt. %, 0.01 wt. %, 0.001 wt. %, or less, e.g., of the total wt. of the composition (in dry form).

In some embodiments, glutamine is absent from the composition, or if present, is present at less than: 10 wt. %, 5 wt. %, 1 wt. %, 0.5 wt. %, 0.1 wt. %, 0.05 wt. %, 0.01 wt. %, 0.001 wt. %, or less, e.g., of the total wt. of the composition (in dry form).

In some embodiments, methionine is absent from the composition, or if present, is present at less than: 10 wt. %, 5 wt. %, 1 wt. %, 0.5 wt. %, 0.1 wt. %, 0.05 wt. %, 0.01 wt. %, 0.001 wt. %, or less, e.g., of the total wt. of the composition (in dry form). In some embodiments, proline is absent from the composition, or if present, is present at less than: 10 wt. %, 5 wt. %, 1 wt. %, 0.5 wt. %, 0.1 wt. %, 0.05 wt. %, 0.01 wt. %, 0.001 wt. %, or less, e.g., of the total wt. of the composition (in dry form). In some embodiments, tryptophan is absent from the composition, or if present, is present at less than: 10 wt. %, 5 wt. %, 1 wt. %, 0.5 wt. %, 0.1 wt. %, 0.05 wt. %, 0.01 wt. %, 0.001 wt. %, or less, e.g., of the total wt. of the composition (in dry form). In some embodiments, one, two, or three of methionine, proline, or tryptophan is absent from the composition, or if present, is present at less than: 10 wt. %, 5 wt. %, 1 wt. %, 0.5 wt. %, 0.1 wt. %, 0.05 wt. %, 0.01 wt. %, 0.001 wt. %, or less, e.g., of the total wt. of the composition (in dry form).

In some embodiments, arginine is absent from the composition, or if present, is present at less than: 10 wt. %, 5 wt. %, 1 wt. %, 0.5 wt. %, 0.1 wt. %, 0.05 wt. %, 0.01 wt. %, 0.001 wt. %, or less, e.g., of the total wt. of the composition (in dry form). In some embodiments, glycine is absent from the composition, or if present, is present at less than: 10 wt. %, 5 wt. %, 1 wt. %, 0.5 wt. %, 0.1 wt. %, 0.05 wt. %, 0.01 wt. %, 0.001 wt. %, or less, e.g., of the total wt. of the composition (in dry form). In some embodiments, arginine and glycine are absent from the composition, or if present, are present at less than: 10 wt. %, 5 wt. %, 1 wt. %, 0.5 wt. %, 0.1 wt.

%, 0.05 wt. %, 0.01 wt. %, 0.001 wt. %, or less, e.g., of the total wt. of the composition (in dry form).

In some embodiments, a carbohydrate (e.g., one, two, three, four, five, six, seven, eight, nine, 10, 11, 12, 13, 14, 15, 16, 17, or 18 of dextrose, maltodextrose, sucrose, dextrin, fructose, galactose, glucose, glycogen, high fructose corn syrup, honey, inositol, invert sugar, lactose, levulose, maltose, molasses, sugarcane, or xylose) is absent from the composition, or if present, is present at less than: 10 wt. %, 5 wt. %, 1 wt. %, 0.5 wt. %, 0.1 wt. %, 0.05 wt. %, 0.01 wt. %, 0.001 wt. %, or less, e.g., of the total wt. of the composition (in dry form).

In some embodiments, a vitamin (e.g., one, two, three, four, five, six, or seven of vitamin B1, vitamin B2, vitamin B3, vitamin B6, vitamin B12, vitamin C, or vitamin D) is absent from the composition, or if present, is present at less than: 10 wt. %, 5 wt. %, 1 wt. %, 0.5 wt. %, 0.1 wt. %, 0.05 wt. %, 0.01 wt. %, 0.001 wt. %, or less, e.g., of the total wt. of the composition (in dry form).

In some embodiments, one or both of nitrate or nitrite are absent from the composition, or if present, are present at less than: 10 wt. %, 5 wt. %, 1 wt. %, 0.5 wt. %, 0.1 wt. %, 0.05 wt. %, 0.01 wt. %, 0.001 wt. %, or less, e.g., of the total wt. of the composition (in dry form).

In some embodiments, 4-hydroxyisoleucine is absent from the composition, or if present, is present at less than: 10 wt. %, 5 wt. %, 1 wt. %, 0.5 wt. %, 0.1 wt. %, 0.05 wt. %, 0.01 wt. %, 0.001 wt. %, or less, e.g., of the total wt. of the composition (in dry form).

In some embodiments, a probiotic (e.g., a Bacillus probiotic) is absent from the composition, or if present, is present at less than: 10 wt. %, 5 wt. %, 1 wt. %, 0.5 wt. %, 0.1 wt. %, 0.05 wt. %, 0.01 wt. %, 0.001 wt. %, or less, e.g., of the total wt. of the composition (in dry form).

In some embodiments, phenylacetate is absent from the composition, or if present, is present at less than: 10 wt. %, 5 wt. %, 1 wt. %, 0.5 wt. %, 0.1 wt. %, 0.05 wt. %, 0.01 wt. %, 0.001 wt. %, or less, e.g., of the total wt. of the composition (in dry form).

In some embodiments, acetyl-L-carnitine is absent from the composition, or if present, is present at less than: 10 wt. %, 5 wt. %, 1 wt. %, 0.5 wt. %, 0.1 wt. %, 0.05 wt. %, 0.01 wt. %, 0.001 wt. %, or less, e.g., of the total wt. of the composition (in dry form).

In some embodiments, gelatin (e.g., a gelatin capsule) is absent from the composition, or if present, is present at less than: 10 wt. %, 5 wt. %, 1 wt. %, 0.5 wt. %, 0.1 wt. %, 0.05 wt. %, 0.01 wt. %, 0.001 wt. %, or less, e.g., of the total wt. of the composition (in dry form).

Methods of Treatment with an Active Moiety to Reduce Fat Infiltration in Muscle

The composition, e.g., Active Moiety, as described herein can be administered to reduce fat infiltration in muscle. Fat infiltration in muscle is an important predictor of muscle function and mobility, independent of as well as associated with other diseases and disorders of muscle atrophy (see, e.g., Addison et al., 2014, Int. J. Endocrinology, dx.doi.org/10.1155/2014/309570) and has been identified as a complication of cirrhosis independently of other co-morbidities, e.g., sarcopenia (see, e.g., Bhanji et al., 2018, Hep. Intl., doi.org/10.1007/s12072-018-9875-9). Fat infiltration has an impact on muscle quality, so identification of a therapeutic that can reduce fat infiltration in muscle is an important development. As shown in the Example, an exemplary composition (e.g., Active Moiety), containing HFKT (using the single amino acid code), showed significant reduction or even avoidance of fat accumulation in muscle cells. This unprecedented observation for this class of active combinations of amino acid entities now identified as Active Moieties opens important new avenues of therapy with an Active Moiety for subjects in need of therapy, as well as methods of monitoring the effect of administration of an Active Moiety.

Thus, a method of the invention includes administration of an effective amount of a composition disclosed herein (e.g., an Active Moiety) to treat subjects at risk of or who have fat infiltration in muscle (i.e., myosteatosis). Myosteatosis is independently associated with end-stage liver disease (ESLD), which is inclusive of, but not limited to, hepatic encephalopathy, variceal bleeding, portal hypertension, ascites, infection risk, sepsis, all-cause hospitalization, and all-cause and liver-related mortality. Other conditions that involve myosteatosis include but are not limited to chronic back pain (fat infiltration in paraspinal muscles); HIV patients (fat infiltration in locomotor muscles); spinal cord injury; stroke; neurological injury; COPD; liver disease or cirrhosis such as hepatic encephalopathy; and muscle weakness associated with ageing.

In addition, lowering muscle fat is a means of improving glucose handling. Given a strong association between insulin resistance and muscle fat, lowering muscle fat improves glucose sensitivity. Glucose disposal is a function of muscle and so lowering fat infiltration in muscle improve a muscle function of sequestering glucose.

Increased infiltration by inter- and intramuscular fat (myosteatosis), in conjunction with reduced muscle mass (myopenia), is recognized as a poor prognostic indicator in patients with cancer (Malietzis et al., Ann Surg. 2016 February; 263(2):320-5). Significantly shorter cancer-specific survival and overall survival times were identified for the myosteatosis versus the non-myosteatosis group in a study of patients who underwent curative colorectal cancer surgery (Sueda et al., Dis Colon Rectum. 2018 March; 61(3):364-374). Myosteatosis, characterized by inter- and intramyocellular fat deposition, is strongly related to poor overall survival after surgery for periampullary cancer (doi.org/10.1016/j.hpb.2018.02.378). Thus, in another embodiment, reduction of fat infiltration in muscle by administration of an effective amount of a composition disclosed herein (e.g., an Active Moiety) improves outcomes for treatment of cancer, such as surgery for colorectal cancer and for periampullary cancer.

Subjects who have increased levels of fat infiltration in muscle may not have significant increase in BMI, sarcopenia, or other overt conditions. Accordingly, the invention provides a method of treating a patient who suffers from cirrhosis without sarcopenia by administering an effective amount of an effective amount of a composition disclosed herein (e.g., an Active Moiety) that reduces fat infiltration in muscle. In another aspect, the invention provides for treating a patient with hepatic encephalopathy by administering an effective amount of an effective amount of a composition disclosed herein (e.g., an Active Moiety) that reduces fat infiltration in muscle.

Patients with rotator cuff injury particularly benefit from the methods of the invention of administering an effective amount of a composition disclosed herein (e.g., an Active Moiety). Fat infiltration in the shoulder muscles is associated with poor outcome from rotator cuff injury (Melis et al., 2009, Orthopaedics & Tramatology: Surgery & Research 95:319-324). Rotator cuff injury includes tendon lesion of the supraspinatus, infraspinatus, and subscapularis. Fat infiltration can occur in any of the affected muscles. Fat infiltration above stage 2 (intermediate) according to the Goutallier classification (see Melis et al.) carries the risk of irreversible functional loss, and a key objective of an intervention during intermediate stages of muscle fatty infiltration is to prevent such permanent loss. Thus, the present invention provides for treating subjects suffering from rotator cuff injury with an effective amount of a composition disclosed herein (e.g., an Active Moiety) to prevent or delay onset of Goutallier Stage 2 fat infiltration, especially prior to surgery to repair the injury. Alternatively, the present invention provides for treating subjects suffering from rotator cuff injury who have Goutallier Stage 2 or greater fat infiltration to reduce the degree of fat infiltration, and thus the Goutallier Stage, prior to surgery. In yet another alternative, the invention provides for treating a subject suffering from a rotator cuff injury with an effective amount of a composition disclosed herein (e.g., an Active Moiety) in addition to medical treatment; medical treatment of rotator cuff injury includes rest, adaptation of daily and occupational movements, rehabilitation, NSAIDs, antalgics, physical therapy, infiltrations, etc. Usually surgery for a rotator cuff injury is surgery to repair a torn tendon, and can be arthroscopic surgery or normal surgery.

The invention is especially useful for treating elderly subjects with a rotator cuff injury, who are at greater risk for more faster fat infiltration and progression to the intermediate Goutallier Stage 2 and beyond to more severe fat infiltration (Stages 3 and 4). Elderly subjects 50 years old or older benefit; subjects 55 years old or older can have even greater benefit; and subjects 60 years old or older even greater benefit than those who are 55 or older, since at each advanced age group susceptibility to fat infusion increases.

Each of the foregoing treatments of rotator cuff injury may include determining the degree of fat infiltration in muscle, e.g., to determine the Goutallier stage; to show changes in Goutallier stage with therapy by administering an effective amount of a composition disclosed herein (e.g., an Active Moiety) to obtain a prognosis of therapy, such as surgery; to establish an appropriate time for surgery; or to determine that surgery is unnecessary.

The present disclosure also provides a method for treating one or more (e.g., all) physiological symptoms selected from immobilization, malnutrition, fasting, aging, autophagy, reduced protein synthesis, anabolic resistance, neuromuscular junction integrity, insulin resistance, decreased mitochondrial biogenesis, anaplerosis, or an energy deficit, in each case along with evaluating fat infiltration in muscle. The method includes administering to a subject in need thereof an effective amount of a composition as set forth hereinabove. Therapeutic treatment according to the invention can be achieved in a subject who has a muscle disease, for example, muscle atrophy, sarcopenia, muscle deterioration, muscle decay, cachexia, drug-induced myopathy, muscular dystrophy, or myopenia. The muscle disease or disorder can be a dystrophy, such as a myotonic dystrophy. For example, the muscle disease or disorder can be DM1.

Alternatively, the muscle disease or disorder can be a drug-induced myopathy, e.g., a statin-induced myopathy; a steroid-induced myopathy; an immunosuppressant-induced myopathy; a chemotherapeutic-induced myopathy; or an alcohol-induced myopathy. In each case, administration of an effective amount of a composition disclosed herein (e.g., an Active Moiety) is accompanied by evaluating fat infiltration in muscle.

In addition, the subject can have a fracture or other trauma other than rotator cuff injury.

In some embodiments, the method includes administering to a subject in need thereof an effective amount of the composition to treat stroke or a neurological injury.

In some embodiments, the method includes administering to a subject in need thereof an effective amount of the composition to treat a food deficiency, e.g., malnutrition or fasting; aging; autophagy; reduced protein synthesis; anabolic resistance; junction integrity (e.g., neuromuscular junction integrity); decreased mitochondrial biogenesis; anaplerosis. In each such foregoing case, treatment is accompanied by evaluating fat infiltration in muscle.

In some embodiments, the subject has not received prior treatment with an effective amount of a composition disclosed herein (e.g., an Active Moiety) (e.g., a naïve subject) accompanied by evaluating fat infiltration in muscle.

In some embodiments, the subject has muscle weakness, e.g., muscle weakness of one, two, or more (e.g., all) of skeletal muscle, cardiac muscle, or smooth muscle. In certain embodiments, the subject has muscle weakness in one, two, three, four, five, six, or more (e.g., all) of a neck muscle, a torso muscle, an arm muscle, a shoulder muscle, a hand muscle, a leg muscle, or a foot muscle. Fat infiltration in muscle can be evaluated daily, every 2-3 days, weekly, every two weeks, every three weeks, and every four weeks after treatment to determine or evaluate the degree of fat infiltration in muscle, particularly to determine that the degree of fat infiltration in muscle is reduced by treatment with an effective amount of a composition disclosed herein (e.g., an Active Moiety). In the case where administration of an effective amount of a composition disclosed herein (e.g., an Active Moiety) precedes an elective procedure, such as orthopedic surgery, evaluating fat infiltration in muscle can be undertaken to determine that the fat fraction in muscle is unchanged from before the surgery, or even improved.

A subject who has had orthopedic surgery, e.g., knee surgery, or hip surgery, elbow surgery, or has worn a cast benefits from administration of an effective amount of a composition disclosed herein (e.g., an Active Moiety) accompanied by evaluating fat infiltration in muscle. When surgery is elective, e.g., for knee or hip replacement (also called total knee arthroplasty and total hip arthroplasty, respectively) administration of the effective amount of a composition disclosed herein (e.g., the Active Moiety), evaluation of fat infiltration in muscle, or both can be done before surgery, e.g., one week before surgery or two weeks before surgery, or can be done after surgery, and preferably is done before and after surgery. In particularly, evaluation of fat infiltration in muscle, specifically muscle most impacted by the surgery or treated by the surgery, can be done daily, every 2-3 days, weekly, every two weeks, every three weeks, and every four weeks after surgery to determine or evaluate the degree of fat infiltration in muscle, particularly to determine that the degree of fat infiltration in muscle is reduced by treatment with an Active Moiety, and more particularly to determine that the fat fraction in muscle is unchanged from before the surgery, or even improved.

In some embodiments, the subject has a neuromuscular disorder, e.g., myasthenia gravis or Lambert-Eaton myasthenic syndrome.

In some embodiments, the subject has muscular dystrophy, e.g., Duchenne muscular dystrophy, Becker muscular dystrophy, facioscapulohumeral muscular dystrophy, or myotonic dystrophy. In some embodiments, the subject has an inflammatory myopathy, e.g., polymyositis or dermatomyositis.

In some embodiments, the subject has one, two, or more (e.g., all) of low sodium levels (e.g., hyponatremia), low potassium levels (e.g., hypokalemia), or a calcium deficiency or relatively high calcium levels (e.g., hypercalcemia).

In some embodiments, the subject has muscle weakness associated with nerve damage, e.g., neuralgia or peripheral neuropathy. In some embodiments, the subject has a bone weakness disease, e.g., osteomalacia, osteogenesis imperfecta, rickets, or hypophosphatasia.

In some embodiments, the subject has experienced a stroke or a transient ischemic attack. In some embodiments, the subject has an autoimmune disease, e.g., Graves' disease.

In some embodiments, the subject has hypothyroidism. In some embodiments, the subject has amyotrophic lateral sclerosis (ALS).

In some embodiments, administering the composition results in activation of muscle protein synthesis in the subject. In some embodiments, the composition also reduces muscle protein wasting.

In some embodiments, the composition results in an improvement in the degree of fat infiltration in muscle associated with one or both of immobilization or muscle disuse following injury in a subject. In some embodiments, the subject has had a surgery, e.g., rotator cuff surgery, knee surgery, or hip surgery, or has worn a cast, prior to administration of the composition. In some embodiments, the subject has had a hip fracture-related myopenia. In some embodiments, the subject has had a joint replacement. In some embodiments, the subject has had an injury repair surgery.

In some embodiments, the subject has ventilator-induced diaphragmatic dystrophy or ventilator-induced diaphragmatic dysfunction. In some embodiments, the subject has had one or both of ICU-acquired or burns-related myopathies.

In some embodiments, the subject has disease-related cachexia, e.g., a disease-related cachexia selected from chronic obstructive pulmonary disease (COPD), congestive heart failure (CHF), chronic kidney disease (CKD), and cancer.

In some embodiments, method of the invention further includes administration of a second agent. Such a second agent may exclude proteins, whether intact or in hydrolyzed form, such as whey, casein, lactalbumin, etc.

The present disclosure also provides a method for reducing muscle atrophy comprising administering to a subject in need thereof an effective amount of a composition described herein. In each such case effectiveness of therapy includes reduction of fat infiltration in muscle and may include determining the degree of fat infiltration in muscle.

The present disclosure also provides a composition described herein for use as a medicament for reducing fat infiltration in muscle.

The present disclosure provides a composition described herein for use as a medicament for reducing fat infiltration in muscle, which may be in conjunction with treating one or more symptoms selected from the group consisting of liver disease, immobilization, injury, surgery, malnutrition, fasting, aging, autophagy, reduced protein synthesis, anabolic resistance, neuromuscular junction integrity, insulin resistance, decreased mitochondrial biogenesis, and anaplerosis.

Treatment with an Active Moiety Accompanied by Evaluating Fat Infiltration in Muscle

In other conditions, administering the composition, e.g., Active Moiety, to improve, e.g., enhance, muscle function, e.g., in a patient with a muscle disease or disorder, includes evaluating fat infiltration in muscle in order to follow treatment progress of, or make a treatment prognosis for, a subject.

As noted above, magnetic resonance imaging (MRI) is a useful method for evaluating fat infiltration in muscle. For example, axial (transverse) images can be obtained from one or both thighs from the distal end of the femur to the greater trochanter using GE high fidelity 3T magnet. A fast-recovery, fast spin echo pulse sequence can be used, along with IDEAL (iterative decomposition of water and fat with echo asymmetry and least-squares estimation) post-processing to obtain water-only, fat-only, in-phase and out-of-phase images of the thigh or thighs. The following parameters may be used: TR=2000 msec, TE=30 msec, refocusing flip angle=111 degrees, echo train length=6, ASSET (parallel imaging factor)=2, field of view=42×21 cm, acquisition matrix=512×256, 3-mm slice thickness, 0-mm slice gap. A total of approximately 160 slices can be acquired, but varied depending on length of the thigh. The acquisition can be done in two sections, a lower stage, and an upper stage. Total scan time for both stages may be approximately 11 minutes. The scans can be uploaded onto Analyze Pro software. The 50% region between the greater trochanter of the hip and lateral epicondyle of the knee can be used for analysis. The segmentation features of the software can be used to differentiate between the bone, fat, right muscle, right quadriceps, left muscle and left quadriceps. Then, every third slice in the 50% region can be manually traced for the quadriceps muscles of both legs. The highest number from these measurements can be taken as the peak quadriceps cross-sectional area. The software is then able to take every third slice that may have been manually measured and extrapolate that data for every slice in the 50% region to get an estimate of quadriceps volume. CSA is generally expressed in mm² and muscle volume in mm³.

To obtain independent verification of the imaging data, DIXON sequences of the upper and lower thighs can be securely transferred to an independent analytical group for whole muscle volume analysis and an additional analysis to measure intramuscular fat fraction. Given the water and fat images it is possible to generate a Fat Fraction (FF) image as:

FF=F/(W+F), where F=fat, and W=water.

These images can be calculated and added to individual studies. As the base images can give spurious regions of high fat fraction due to noise, a thresholding filter can be used to reduce these small peripheral artefacts and minimize noise in regions where both the fat and water signals are small.

### Dosage Regimens

The composition (e.g., the Active Moiety) can be administered to a human subject according to a dosage regimen described herein. In some embodiments, EAAs (e.g., one, two, or three of a histidine, histidine amino acid entity, and threonine) are included in the composition at a close to achieve stoichiometry with the level of AAAs (e.g., one or both of tyrosine or phenylalanine) in a subject.

Doses can be administered, e.g., twice daily, three times daily, four times daily, five times daily, six times daily, seven times daily, or more. The composition can be administered for at least 2 days, 3 days, 4 days, 5 days, 6 days, 7 days, or 2 weeks. Depending on the condition being treated, the composition can be administered for at least 10 weeks, 11 weeks, 12 weeks, 13 weeks, 14 weeks, 15 weeks, 16 weeks, 17 weeks, 18 weeks, 19 weeks, 20 weeks, or longer at a dose of 5 g+/−20% g daily to 100 g+/−20% g daily, e.g., 10 g+/−20% g daily to 75 g+/−20% g daily. In some embodiments, the composition is administered at a dosage of 10 g+/−20% g daily, 15 g+/−20% g daily, 20 g+/−20% g daily, 25+/−20% g daily, 30+/−20% g daily, 35+/−20% g daily, 40+/−20% g daily, 41+/−20% g daily, 42+/−20% g daily, 43+/−20% g daily, 44+/−20% g daily, 45+/−20% g daily, 46+/−20% g daily, 47+/−20% g daily, 48+/−20% g daily, 49+/−20% g daily, 50+/−20% g daily, 55+/−20% g daily, or 60+/−20% g daily. In certain embodiments, the composition is administered at a dosage of 44+/−20% g daily.

In some embodiments, the composition is administered with a meal. In some embodiments, the composition is administered between meals, e.g., before or after a meal. In some embodiments, the composition is administered at least once during the day and at least once in the late evening or before bedtime. In some embodiments, the composition can be administered chronically, e.g., more than 30 days, e.g., 31 days. 40 days, 50 days, 60 days, 3 months, 6 months, 9 months, one year, two years, or three years). If the condition of fat infiltration in muscle is chronic and unremitting, the invention contemplates administering a composition (e.g., an Active Moiety) indefinitely, e.g., for the life of the subject.

The Active Moiety can be administered at a dose of about 4 g and about 80 g total amino acids, e.g., once per day, twice per day, three times per day, four times per day, five times per day, or six times per day (e.g., three times per day). In some embodiments, the composition is administered at a dose of about 5 g to about 15 g, about 10 g to about 20 g, about 20 g to about 40 g, or about 30 g to about 50 g total amino acids, e.g., once per day, twice per day, three times per day, four times per day, five times per day, or six times per day (e.g., three times per day).

The composition can be administered every hour, every 2 hours, every 3 hours, every 4 hours, every 5 hours, every 6 hours, every 7 hours, every 8 hours, every 9 hours, every 10 hours, every 11 hours, every 11 hours, every 12 hours, every 13 hours, every 14 hours, or every 16 hours while the patient is awake to enhance muscle function in a subject (e.g., the subject has or is identified as having decreased muscle function due to aging, injury, atrophy, infection, or disease).

In some embodiments, the composition is administered prior to a meal (e.g., one, two, or more (e.g., all) of breakfast, lunch, or dinner). In some embodiments, the composition is administered conccurrent with a meal (e.g., one, two, or more (e.g., all) of breakfast, lunch, or dinner). In some embodiments, the composition is administered following a meal (e.g., one, two, or more (e.g., all) of breakfast, lunch, or dinner). In an embodiment, one dose of the composition is administered in the late evening.

In some embodiments, the composition comprises three stick packs, e.g., each stick pack comprising 33.3%+/−20% of the quantity of each amino acid entity included in the composition described herein. In certain embodiments, three stick packs are administered three times daily.

In some embodiments, the composition is administered at a dose of 2 g+/−20% to 60 g+/−20% total amino acid entities, e.g., once daily, twice daily, three times daily, four times daily, five times daily, or six times daily (e.g., three times daily). In some embodiments, the composition is administered at a dose of 2 g+/−20% to 10 g+/−20%, 10 g+/−20% to 40 g+/−20%, or 40 g+/−20% to 60 g+/−20% total amino acid entities, e.g., once daily, twice daily, or three times daily (e.g., three times daily). In certain embodiments, the composition is administered at a dose of 10 g+/−20% to 40 g+/−20% total amino acid entities twice daily, e.g., 10 g+/−20%, 15 g+/−20%, 20 g+/−20%, 25 g+/−20%, 30 g+/−20%, 35 g+/−20%, or 40 g+/−20% total amino acid entities three times daily (e.g., 15 g+/−20%).

In some embodiments, the composition can be administered to a subject with a carbohydrate supplement, e.g., when administered in the night, late evening, or before bedtime (Table 7). In some embodiments, the composition, when administered in the late evening or before bedtime, further includes at least 50 kcal, at least 100 kcal, or at least 200 kcal of carbohydrate supplement for nocturnal dosing. In some embodiments, the carbohydrate supplement is administered at a dose of 30 g+/−20% to 90 g+/−20% (e.g. 55 g+/−20%) in the late evening with the composition. In some embodiments, the carbohydrate supplement can include a polysaccharide (e.g., maltodextrin (e.g., 50+/−20% g of maltodextrin)) and a fermentable fiber or prebiotic (e.g., one or both of beta-glucan (e.g., 2.5+/−20% g of beta-glucan) or resistant starch (e.g., 2.5+/−20% g of resistance starch)). In some embodiments, the carbohydrate supplement can be provided in a powder or liquid form and mixed with the composition for administration (e.g., at night) to a subject. In some embodiments, administration of the composition with the carbohydrate supplement supports overnight anabolic metabolism in a subject.

TABLE 7
Exemplary carbohydrate supplement for
administration with the composition.

| Compo- | | Est | Est | Exemplary Function in Composi- |
|---|---|---|---|---|
| nent | Grams | kcal/g | kcal | tion |
| Malto- | 50 | 4 | 200 | Polyaccharide as a major source of |
| dextrin | | | | glucose and caloric content. |
| Beta- | 2.5 | 4 | 10 | Fermentable fiber/prebiotic to slow |
| glucan | | | | absorption of glucose |
| | | | | (from maltodextrin), provide |
| | | | | additional caloric content, and |
| | | | | promote restorative changes in gut |
| | | | | microbiota. |
| Resistant | 2.5 | 2 | 5 | Fermentable fiber/prebiotic to slow |
| Starch | | | | absorption of glucose (from |
| | | | | maltodextrin), |
| Total | 55 | | | provide additional caloric content, |
| carbs/day | | | | and promote restorative changes in |
| Estimated | | | 215 | gut microbiota. |
| cal/day | | | | |

### Production of Active Moiety and Pharmaceutical Compositions

Amino acids used to make the compositions (e.g., an Active Moiety) may be agglomerated, and/or instantized to aid in dispersal and/or solubilization.

The amino acid compositions of the present disclosure may be made using amino acids and amino acid derivatives from the following sources, or other sources may be used: e.g., FUSI-BCAA™ Instantized Blend (L-Leucine, L-Isoleucine and L-Valine in 2:1:1 weight ratio), instantized L-Leucine, and other acids may be obtained from Ajinomoto Co., Inc. Pharma. grade amino acid entity raw materials may be used in the manufacture of pharmaceutical amino acid entity products. Food (or supplement) grade amino acid entity raw materials may be used in the manufacture of dietary amino acid entity products.

To produce the amino acid compositions of the instant disclosure, the following general steps may be used: the starting materials (individual amino acids and excipients) may be blended in a blending unit, followed by verification of blend uniformity and amino acid content, and filling of the blended powder into stick packs or other unit dosage form. The content of stick packs or other unit dosage forms may be dispersed in water at time of use for oral administration. See U.S. patent application Ser. No. 16/446,171, filed Jun. 19, 2019, entitled “METHODS OF MANUFACTURING AMINO ACID COMPOSITIONS,” which is incorporated herein by reference in its entirety.

Pharmaceutical compositions of the present disclosure may be in a form suitable for oral use (for example aqueous or oily suspensions, emulsions, dispersible powders or granules, syrups or elixirs), for parental administration (for example as a sterile aqueous or oily solution for intravenous, subcutaneous, intramuscular dosing or as a suppository for rectal dosing) or for enteral administration (for example via tube feeding). Generally, however, pharmaceutical compositions of the invention will be for oral administration.

Food supplement and medical nutrition compositions of the invention will be in a form suitable for oral administration.

When combining raw materials, e.g., pharmaceutical grade amino acid entities and/or excipients, into a composition, contaminants may be present in the composition. A composition meets a standard for level of contamination when the composition does not substantially comprise (e.g., comprises less than 10, 9, 8, 7, 6, 5, 4, 3, 2, 1, 0.1, 0.01, or 0.001% (w/w on a dry weight basis)) a contaminant. In some embodiments, a composition described in a method herein does not comprise a contaminant. Contaminants include any substance that is not deliberately present in the composition (for example, pharmaceutical grade amino acid entities and excipients, e.g., oral administration components, may be deliberately present) or any substance that has a negative effect on a product quality parameter of the composition (e.g., side effects in a subject, decreased potency, decreased stability/shelf life, discoloration, odor, bad taste, bad texture/mouthfeel, or increased segregation of components of the composition). In some embodiments, contaminants include microbes, endotoxins, metals, or a combination thereof. In some embodiments, the level of contamination, e.g., by metals, lecithin, choline, endotoxin, microbes, or other contaminants (e.g., contaminants from raw materials) of each portion of a composition is below the level permitted in food.

### Excipients

The amino acid compositions of the present disclosure may be compounded or formulated with one or more excipients. Non-limiting examples of suitable excipients include a tastant, a flavorant, a buffering agent, a preservative, a stabilizer, a binder, a compaction agent, a lubricant, a dispersion enhancer, a disintegration agent, a flavoring agent, a sweetener, and a coloring agent. U.S. patent application Ser. No. 16/446,192, filed Jun. 19, 2019, entitled “COMPOSITIONS FOR THERAPY AND HEALTH CONTAINING AMINO ACIDS WITH BITTER TASTE,” which is incorporated herein by reference in its entirety describes suitable excipients for inclusion in compositions of the invention.

In some embodiments, the excipient comprises a buffering agent. Non-limiting examples of suitable buffering agents include citric acid, sodium citrate, magnesium carbonate, magnesium bicarbonate, calcium carbonate, and calcium bicarbonate.

In some embodiments, the excipient comprises a preservative. Non-limiting examples of suitable preservatives include antioxidants, such as alpha-tocopherol and ascorbate, and antimicrobials, such as parabens, chlorobutanol, and phenol.

In some embodiments, the composition comprises a binder as an excipient. Non-limiting examples of suitable binders include starches, pregelatinized starches, gelatin, polyvinylpyrolidone, cellulose, methylcellulose, sodium carboxymethylcellulose, ethylcellulose, polyacrylamides, polyvinyloxoazolidone, polyvinylalcohols, C12-C18 fatty acid alcohol, polyethylene glycol, polyols, saccharides, oligosaccharides, and combinations thereof.

In some embodiments, the composition comprises a lubricant as an excipient. Non-limiting examples of suitable lubricants include magnesium stearate, calcium stearate, zinc stearate, hydrogenated vegetable oils, sterotex, polyoxyethylene monostearate, talc, polyethyleneglycol, sodium benzoate, sodium lauryl sulfate, magnesium lauryl sulfate, and light mineral oil.

In some embodiments, the composition comprises a dispersion enhancer as an excipient. Non-limiting examples of suitable dispersants include starch, alginic acid, polyvinylpyrrolidones, guar gum, kaolin, xanthan gum, bentonite, purified wood cellulose, sodium starch glycolate, isoamorphous silicate, and microcrystalline cellulose as high HLB emulsifier surfactants.

In some embodiments, the composition comprises a disintegrant as an excipient. In some embodiments, the disintegrant is a non-effervescent disintegrant. Non-limiting examples of suitable non-effervescent disintegrants include starches such as corn starch, potato starch, pregelatinized and modified starches thereof, sweeteners, clays, such as bentonite, micro-crystalline cellulose, alginates, sodium starch glycolate, gums such as agar, guar, locust bean, karaya, pectin, and tragacanth. In some embodiments, the disintegrant is an effervescent disintegrant. Non-limiting examples of suitable effervescent disintegrants include sodium bicarbonate in combination with citric acid, and sodium bicarbonate in combination with tartaric acid.

In some embodiments, the excipient comprises a flavoring agent. Flavoring agents can be chosen from synthetic flavor oils and flavoring aromatics; natural oils; extracts from plants, leaves, flowers, and fruits; and combinations thereof. In some embodiments, the flavoring agent is selected from cinnamon oils; oil of wintergreen; peppermint oils; clover oil; hay oil; anise oil; eucalyptus; vanilla; citrus oil such as lemon oil, orange oil, grape and grapefruit oil; and fruit essences including apple, peach, pear, strawberry, raspberry, cherry, plum, pineapple, and apricot.

In some embodiments, the excipient comprises a sweetener. Non-limiting examples of suitable sweeteners include glucose (corn syrup), dextrose, invert sugar, fructose, and mixtures thereof (when not used as a carrier); saccharin and its various salts such as the sodium salt; dipeptide sweeteners such as aspartame; dihydrochalcone compounds, glycyrrhizin; Stevia Rebaudiana (Stevioside); chloro derivatives of sucrose such as sucralose; and sugar alcohols such as sorbitol, mannitol, xylitol, and the like. Also contemplated are hydrogenated starch hydrolysates and the synthetic sweetener 3,6-dihydro-6-methyl-1,2,3-oxathiazin-4-one-2,2-dioxide, particularly the potassium salt (acesulfame-K), and sodium and calcium salts thereof.

In some embodiments, the composition comprises a coloring agent. Non-limiting examples of suitable color agents include food, drug and cosmetic colors (FD&C), drug and cosmetic colors (D&C), and external drug and cosmetic colors (Ext. D&C). The coloring agents can be used as dyes or their corresponding lakes.

Particular excipients may include one or more of: citric acid, lecithin, (e.g., Alcolec F100), sweeteners (e.g., sucralose, sucralose micronized NF, acesulfame potassium (e.g., Ace-K)), a dispersion enhancer (e.g., xanthan gum (e.g., Ticaxan Rapid-3)), flavorings (e.g., vanilla custard #4306, Nat Orange WONF #1326, lime 865.0032U, and lemon 862.2169U), a bitterness masking agent (e.g., 936.2160U), and natural or artificial colorings (e.g., FD&C Yellow 6). Table 12 in the Examples illustrates a formulation with such excipients. Exemplary ingredient contents for each stick pack are shown in Table 8.

TABLE 8
Exemplary ingredient contents in each stick pack.

| INGREDIENT | GRADE | FUNCTION |
|---|---|---|
| Amino Acids | USP | Active Pharmaceutical |
| | | Ingredient (API) |
| Citric Acid | USP | pH, Flavor |
| Acesulfame K | NF | Sweetness (rapid onset) |
| Sucralose | NF | Sweetness (slow onset) |
| Lecithin (Alecolec F100) | FCC | Wetting Agent |
| Xanthan Gum | FCC | Stabilizer/Thickener |
| Vanilla Custard (Art) | GRAS | Taste/Aroma |
| Orange (Natural and | GRAS | 1° flavor |
| WONF) | | |
| Lime (Natural and WONF) | GRAS | 2° flavor |
| Lemon (Natural and | GRAS | 2° flavor |
| artificial) | | |
| Taste Modifier | GRAS | Bitterness masking |
| FD&C Yellow No. 6 | USP | Color |

In another embodiment, excipients are limited to citric acid, a sweetener (e.g., sucralose), xanthan gum, an aroma agent (e.g., vanilla custard #4036), a flavoring agent (e.g., Nat orange WONF #1362), and a coloring agent (e.g., FD&C Yellow 6), e.g., the excipient specifically excludes lecithin (Table 9).

TABLE 9
Exemplary contents in each stick pack.

| INGREDIENT | GRADE | FUNCTION |
|---|---|---|
| Amino Acids | USP | Active Pharmaceutical |
| | | Ingredient (API) |
| Citric Acid | USP | pH, Flavor |
| Sucralose | NF | Sweetness (slow onset) |
| Xanthan Gum | FCC | Stabilizer/Thickener |
| Vanilla Custard (Art) | GRAS | Aroma |
| Orange (Nat + WONF) | GRAS | 1° flavor |
| FD&C Yellow No. 6 | USP | Color |

### Dietary Compositions

The Active Moiety including amino acid entities can be formulated and used as a dietary composition, e.g., chosen from a medical food, a functional food, or a supplement. In such an embodiment, the raw materials and final product should meet the standards of a food product. Such uses include improving health of a subject having or identified as suffering from infiltration of fat in muscle due to aging, injury, atrophy, infection, or disease. In some embodiments, the subject has or is identified as having muscle deterioration, muscle decay, muscle atrophy, cachexia, sarcopenia, steroid myopathy, or muscular dystrophy. In some embodiments, the subject has one or both of type 2 diabetes or a relatively high BMI.

In some embodiments, administration of the dietary composition results in an improvement in one or more metabolic symptoms in the subject, e.g., one or more metabolic symptoms is selected from the following: increased free fatty acid and lipid metabolism, improved mitochondrial function, white adipose tissue (WAT) browning, decreased reactive oxygen species (ROS), increased levels of glutathione (GSH), decreased hepatic inflammation, decreased hepatocyte ballooning, improved gut barrier function, increased insulin secretion, or glucose tolerance. In certain embodiments, administration of the composition results in an improvement in one or more metabolic symptoms after a treatment period of 24 hours.

### Biomarkers

Any of the methods disclosed herein can include evaluating or monitoring the effectiveness of administering a composition described herein to a subject by determining the degree of infiltration of fat in muscle in the subject, e.g., with CT or MRI. The subject may be in need of muscle function enhancement (e.g., a subject having muscle deterioration, muscle decay, muscle atrophy, cachexia, sarcopenia, drug-induced myopathy, muscular dystrophy, or myopenia). The effectiveness to the composition in treating a subject can further comprise a measure of the levels of one or more (e.g., all) of the following:
a) myostatin;
b) myoglobin;
c) Cortisol-AM;
d) C-reactive protein;
e) insulin;
f) cytokines (e.g., one or more (e.g., all) of IL-1A RBM, IL-1RA, IL-1 RI, IL-1 RII, IL-12, IL-18, or MCP-1);
g) GDF-11;
h) P3NP;
i) IGF-1;
j) IGFBP1;
k) IGFBP3;
l) FGF21;
m) DHEAS;
n) mTORC1;
o) Gcn2; or
p) AMP-activated protein kinase (AMPK).

In some embodiments of any of the methods disclosed herein, the measure of one or more of a)-p) is obtained from a sample acquired from the subject.

In some embodiments, the subject is evaluated prior to receiving, during, or after receiving, the composition.

In some embodiments, administration of the composition to the subject results in a decrease in levels of one or more (e.g., all) of myoglobin, myostatin, GDF-11, cortisol-AM, C-reactive protein, insulin, or cytokines (e.g., one or more (e.g., all) of IL-1A RBM, IL-1RA, IL-1 RI, IL-1 RII, IL-12, IL-18, or MCP-1) in the subject (Table 10). In some embodiments, administration of the composition to the subject results in an increase in levels of one or more (e.g., all) of P3NP, IGF-1, IGFBP1, IGFBP3, FGF-21, DHEAS, or mTORC1 in the subject (Table 10).

TABLE 10
Additional biomarkers to determine effect
of the composition on muscle biology.

| | | Expected | |
|---|---|---|---|
| | | Change in | Additional information |
| | | Response | regarding biomarker change |
| | | to Com- | on muscle synthesis and/or |
| Biomarker | Category | position | breakdown |
| Myoglobin | Muscle | Down | Decrease suggests a reduction |
| | biology | | in muscle breakdown and |
| | | | autophagy |
| Myostatin, | Muscle | Down | Myostatin act to inhibit |
| GDF-11 | biology | | muscle synthesis decrease |
| | | | in levels indicate increase |
| | | | anabolism Change in GDF-11 |
| | | | levels to further inform |
| | | | changes to muscle biology |
| P3NP | Muscle | Up | P3NP is released during |
| | biology | | collagen synthesis in muscle |
| | | | Increased circulating P3NP |
| | | | indicates muscle growth, |
| | | | muscle repair and fibrosis |
| Cortisol-AM | Endocrine | Down | Endocrine molecules |
| C-reactive | Endocrine | Down | involved in regulating |
| protein | | | protein synthesis as |
| IGF-1, | Endocrine | Up | stimulators/potentiators or |
| IGFBP1, | | | inhibitors |
| IGFBP3, | | | Increase in potentiator |
| FGF21, | | | levels and decrease in |
| DHEAS | | | inhibitor levels are |
| | | | supportive of net anabolism |
| Insulin | Endocrine | Down | Decrease indicates moderation |
| | (glucose | | in insulin resistance, and |
| | tolerance) | | increased glucose handling |
| | | | and anabolic sensitivity |
| IL1ARBM, | Inflamma- | Down | Increased muscle wasting is |
| IL1RA, IL1RI, | tion | | associated with a strong |
| IL1RII, IL-12, | | | inflammatory response |
| IL-18, MCP-1, | | | Reduced levels of these |
| cytokines | | | inflammation biomarkers |
| | | | indicate reduction in |
| | | | inflammation |
| | | | Overall profile of these |
| | | | biomarker can further provide |
| | | | dynamic assessment on |
| | | | interleukin response to the |
| | | | composition |

### EXAMPLES

The Examples below are set forth to aid in the understanding of the invention, but are not intended to, and should not be construed to, limit its scope in any way.

### Example 1. Evaluation of Muscle Fat Accumulation in Myocytes In Vitro

Muscle fat infiltration appears to be a key risk factor for morbidity across a number of complex disease states. The ability of amino acids to influence lipid accumulation in muscle cells was assessed using a modified version of MyoScreen (Cytoo SA, Grenoble Fr).

### Cell Seeding and Differentiation

Primary human myoblasts (from a single donor) were amplified and seeded into CYTOOplates on Day 0. After one day differentiation was started in DMEM/F12 +GlutaMAX supplemented with 0.1% horse serum.

### Culture Conditions and Different Amino Acids Combinations

After 4 days the differentiated myotubes are washed and culture conditions are changed to DMEM 200 uM Free Fatty Acids 2:1 Oleate:Palmitate, 10 ng/mL TNFα, high glucose, 100 μM carnitine, and a poor amino acid media designed to be half concentration of a composite sarcopenic plasma profile, physiologically intended reflects an elderly frail condition. Additionally, cells were untreated or treated with combinations of essential amino acids, either Leucine Isoleucine Valine (LIV) or Histidine Lysine Threonine Phenylalanine (HKFT) at concentrations of 1×, 2×, and 5× the plasma values of healthy humans.

Intracellular Lipid Accumulation Analysis after 4 Days by Fluorescence Microscopy

After 4 days of culture cells were washed twice with PBS 1× (Gibco), fixed with 4% Paraformaldehyde, and washed twice with PBS 1× (100 ul). After fixation cells are immune-stained for troponin to identify specific myotubes. Lipids were stained with HCS LipidTOX Neutral lipid stain (Thermofisher Scientific) diluted 1000× and nuclei were stained with Hoechst 3342 (Life Technologies) diluted to 4 ug/ml. The LipidTOX™ neutral lipid stain has an extremely high affinity for neutral lipid droplets that was detected by fluorescence microscopy using a high content imager (Molecular Devices). LipidTOX signal was restricted to the myotube mask generated by the troponin co-stain. Data are collected as density of lipid droplets (nb/mm²) and mean intensity (au) Images were acquired with Operetta HCS platform (Perkin Elmer) using a 10× objective in three fluorescent channels: nuclei, Troponin T, and LipidTOX. Image processing and analysis are performed with a dedicated algorithm developed on Acapella high Content imaging Software (Perkin Elmer) at CYTOO. Statistical analysis is done with T-Test relative to the untreated condition.

### Results

### Lipid Accumulation and Steatosis Phenotypes

Table 11 shows lipid drop let density and lipid content intensity to describe total lipid content of the myotubes. Treatment with LIV results in a statistically significant dose-dependent increase in both lipid droplet density and lipid intensity. Conversely, HKFT significantly decreases lipid accumulation in a dose-dependent manner. Log 2FC represents the Log 2-transformed fold change from the untreated condition. Positive values indicate that lipid accumulation increased and negative values indicated that lipid accumulation decreased from baseline.

TABLE 11
Changes in lipid accumulation with EAA treatments

| | | | Mean | Median | | |
|---|---|---|---|---|---|---|
| Phenotype | Treatment | Dose | (log2FC) | (log2FC) | std | p-value |
| Spot density (nb/mm²) | HKFT | 1 | −0.012 | −0.009 | 0.079 | 0.75660 |
| Spot density (nb/mm²) | HKFT | 2 | −0.123 | −0.123 | 0.070 | 0.01695 |
| Spot density (nb/mm²) | HKFT | 5 | −0.421 | −0.401 | 0.089 | 0.00045 |
| Spot density (nb/mm²) | LIV | 1 | 0.236 | 0.221 | 0.066 | 0.00135 |
| Spot density (nb/mm²) | LIV | 2 | 0.299 | 0.287 | 0.072 | 0.00076 |
| Spot density (nb/mm²) | LIV | 5 | 0.420 | 0.381 | 0.110 | 0.00102 |
| Mean intensity in myotubes (au) | HKFT | 1 | −0.017 | −0.013 | 0.076 | 0.63378 |
| Mean intensity in myotubes (au) | HKFT | 2 | −0.004 | 0.007 | 0.058 | 0.89782 |
| Mean intensity in myotubes (au) | HKFT | 5 | −0.103 | −0.117 | 0.064 | 0.02292 |
| Mean intensity in myotubes (au) | LIV | 1 | 0.126 | 0.106 | 0.052 | 0.00559 |
| Mean intensity in myotubes (au) | LIV | 2 | 0.236 | 0.239 | 0.017 | 0.00001 |
| Mean intensity in myotubes (au) | LIV | 5 | 0.297 | 0.311 | 0.087 | 0.00160 |

### Example 2. In Vitro Culture of Primary Human Differentiated Myoblasts in Medium Designed to Replicate a Sarcopenic Plasma Profile and Tested for Lipid Accumulation

The disclosure provides assays to evaluate reduction of fat accumulation in myocytes cultured in vitro, e.g., with primary human differentiated myoblasts in medium designed to replicate a sarcopenic plasma profile and tested for lipid accumulation, e.g., in the exemplary protocol described herein. Primary human myoblasts are amplified and seeded onto plates on Day 0. After one day differentiation was started, e.g., with DMEM/F12+GlutaMAX supplemented with 0.1% horse serum. After culture, e.g., 4 days, the differentiated myotubes are washed and culture conditions are changed to challenge conditions, such as DMEM 200 uM Free Fatty Acids 2:1 Oleate:Palmitate, 10 ng/mL TNFα, high glucose, 100 μM carnitine, and a poor amino acid media designed to be half concentration of a composite sarcopenic plasma profile, physiologically intended reflects an elderly frail condition. Additionally, cells were untreated or treated with test compositions, e.g., combinations of essential amino acids, either Leucine Isoleucine Valine (LIV) or Histidine Lysine Threonine Phenylalanine (HKFT) at concentrations of 1×, 2×, and 5× the plasma values of healthy humans. After culture under the test conditions, e.g., 4 days of culture, cells are washed, fixed, e.g., with 4% Paraformaldehyde, and washed twice more. After fixation cells can be stained to identify specific myotubes, e.g., immune-stained for troponin. Lipids can be stained with a lipid stain, e.g., HCS LipidTOX Neutral lipid stain (Thermofisher Scientific) diluted 1000×, and nuclei can be stained with a nuclear stain, e.g., Hoechst 3342 (Life Technologies) diluted to 4 μg/mL.

### Example 3. Clinical Study

### Overview

### 1.1 Rationale for the Study

Response of plasma ammonia and key structural (lean tissue mass) and functional (strength, balance) changes to a concurrent administration of a defined mixture of amino acids and a protein meal in subjects with mild to moderate hepatic insufficiency was previously evaluated a detailed assessment of the safety, tolerability, and physiological response to different amounts of CS-EMM-001 (14.7 g/day and 44.1 g/day for 15 days each), which assessed standard safety chemistry, hematology, ammonia, lean mass, strength and balance measurements in a monitored, domiciled setting in Childs A and B subjects. This study was a 2-period crossover study conducted in 2 parts. In Part 1, only a standard protein shake was administered. Part 2 was a 2-period crossover (each period was of 15-day duration) where subjects were domiciled in a clinical research unit for the 15-day Administration Period. In Period 1, subjects were assigned to receive AXA1665 44.1 g/day administered in three divided amounts for 15 consecutive days, or a bedtime snack only. In Period 2, subjects that had received AXA1665 in Period 1 crossed-over to receive a bedtime snack, and those that received the bedtime snack crossed-over to receive AXA1665 14.7 g/day in three divided amounts for 15 consecutive days. There was a 14-day washout period in between periods where subjects were not administered any study product and were at home.

Daily consumption of CS-EMM-001 at 14.7 or 44.1 g/day for 15 days each was found to be safe and well tolerated, with supraphysiological changes in plasma concentration of the constituent amino acids within CS-EMM-001. These changes in amino acids resulted in a corresponding increase in the basal (fasting) Fischer's (BCAA:AAA) and the Val:Phe ratios by Day 15 as compared to Day 1. Subjects consuming the higher amounts of CS-EMM-001 (44.1 g/day) tended to maintain a leaner phenotype with a lowered liver frailty index (LFI) score.

There were no clinically significant changes in laboratory parameters, liver function tests, blood urea nitrogen or creatinine in any subject during this study. Of note, the percent change from baseline in serum albumin trended higher after consumption of CS-EMM-001 at 44.1 g/day (but not at 14.7 g/day) at the end of the 15-day administration period relative to the corresponding control group, suggesting a directional change toward improvement in nutritional status. Other laboratory values during CS-EMM-001 consumption were similar to those for the same subjects during the control period.

Since it has been well established that consumption of protein rich meals, including mixtures of amino acids in subjects with any degree of hepatic insufficiency transiently raises plasma ammonia by as much as 40% (Dam et al., 2011; Campollo et al., 2017), ammonia was measured as part of the safety laboratory measures. Administration of the 35 g protein shake alone (in Part 1 of the study) resulted in a substantial increase in plasma ammonia over the 5-hour sampling period, consistent with the published literature. CS-EMM-001 alone did not increase ammonia levels despite the administration of added nitrogen load (via exogenous amino acids), and the magnitude of ammonia change seen in the 44.1 g/day group was similar to that observed in the control group. These data therefore suggest an ammonia dampening effect of CS-EMM-001.

The current study builds upon the preceding study with the primary intension to further investigate CS-EMM-001's impact on safety, tolerability, structure (e.g., lean mass, intramuscular fat) and function (e.g., strength, balance, gait speed) assessments over a longer duration (12 weeks), at slightly higher amounts (53.9 g/day), including a lower amount (29.4 g/day), to discern any responsiveness in above parameters to amount administered in a population of mild and moderate hepatic insufficiency with underlying sarcopenia.

### 1.2 Study Population

Current epidemiological data in subjects with chronic liver disease suggest that amino acid physiology is most perturbed in those with muscle loss and dysfunction, with the latter being a key effect modifier of complications commonly seen in this population, such as HE. Muscle depletion and muscle fat infiltration independently increased the risk of both overt and minimal HE with increased mortality (Merli et al, 2013), including increasing the risk of other cirrhosis-related complications, such as ascites, infections, and liver-related hospitalizations (Lucero, et al. 2015). A recent systemic review and meta-analysis comprising 1795 patents showed that sarcopenia was positively associated with the presence of HE (OR 2.74 with a 95% CI, 1.87 to 4.01). The presence of inter- and intramuscular adipose tissue (i.e., myosteatosis) has also been found to adversely impact metabolism and peak force generation, in part via increased inflammatory cytokines in the local environment, altered myokine secretion, and impaired blood flow to the muscle occurring in the presence of myosteatosis. Hence, myosteatosis could be associated with HE by impairing muscle function (force production, metabolism, thermoregulation, and myokine production), irrespective of muscle mass (Correa-de-Araujo et al 2010). Consistent with this notion, myosteatosis was found to be independently associated with HE after adjusting for sarcopenia and model for end-stage liver disease (MELD) score and in increasing mortality in patients with cirrhosis (Kalafateli et al 2018). For these reasons, it is important to thoroughly characterize the structural and functional changes, particularly on myosteatosis, induced by amino acid ingestion in the Childs A and B population.

2 OBJECTIVES

This study's aim is to examine the safety, tolerability, and physiological regulation of a proprietary amino acid food product, CS-EMM-001, over 12 weeks in subjects with mild and moderate hepatic insufficiency.

### 2.1 Study Assessments

Safety and Tolerability Assessments:
Reported AEs, including worsening of HE or any clinically significant liver-related events such as hepatic decompensation, hospitalizations for liver-related complications;
Laboratory assessments (standard chemistry, hematology, urine analysis, plasma ammonia, amino acids, metabolic and inflammation markers);
Vital sign measurements;
Electrocardiograms (ECGs);
Physical and neurological examination;
Body weight, body mass index, waist circumference and mid-arm muscle circumference.

Physiological Assessments of Normal Structure and Function:

Structural Changes by Magnetic Resonance Imaging (MRI) Over 12 Weeks:
Total muscle volume of each thigh;
Cross-sectional area of each thigh;
Cross-sectional area of paraspinal and abdominal muscles at level of the third lumbar vertebra (L3);
Muscle fat fraction within each thigh;
Muscle fat fraction within all muscles at L3;
Body composition profile including visceral fat, subcutaneous fat, liver fat, muscle fat, total abdominal fat index and lean muscle tissue.

Functional Changes Over 12 Weeks:
Overall activity and sleep changes measured by actigraphy;
Physical function assessed by gait speed, and liver frailty index (LFI: hand grip strength, chair stand, balance assessments);
Psychological function assessed psychometric hepatic encephalopathy score (PHES), critical flicker frequency (CFF), and Stroop tests;
Chronic Liver Disease Questionnaire (CLDQ).

3 Overall Study Design

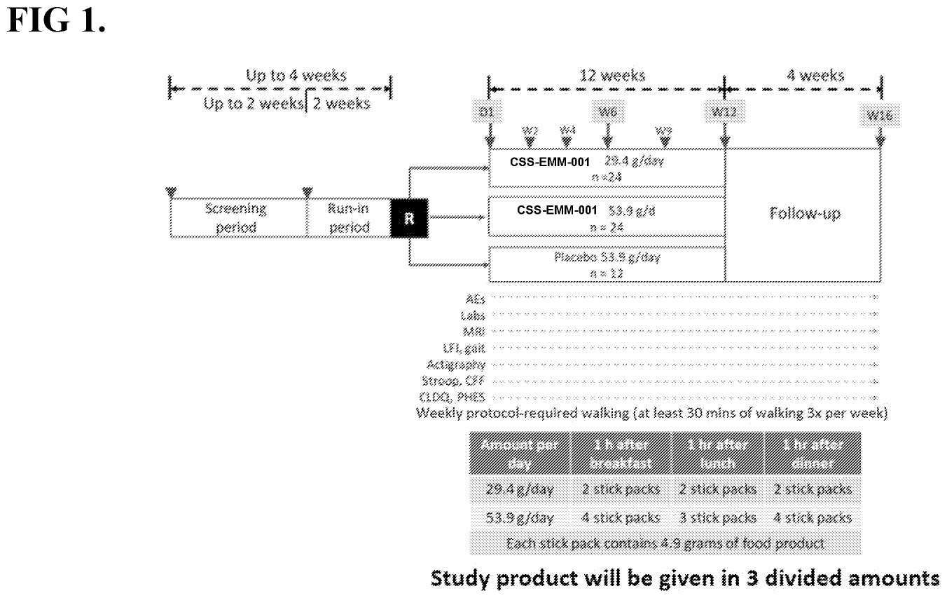

This is a 12-week, randomized, single-blind, placebo-controlled study to assess the safety and tolerability of CS-EMM-001 subjects with mild and moderate hepatic insufficiency (i.e. Child-Pugh class A and B). Approximately 60 subjects are enrolled into this study. A schedule of assessments is presented in FIG. 1.

### 3.1 Screening

Following completion of informed consent procedures, subjects will enter the Screening Period. All screening assessments are conducted, and eligibility confirmed, within 2 weeks prior to the Run-in Visit. Screening re-tests are permitted.

### 3.2 Run-in Period

Once eligibility has been confirmed, subjects enter a Run-in Period that occurs approximately 2 weeks prior to Day 1 and the start of the Administration Period. At the Run-in Visit (Visit 2), subjects are provided with standard-of-care nutrition and activity guidelines for hepatic insufficiency and encouraged to incorporate aspects of these recommendations to the best of their ability into their daily routines for the duration of the study.

### 3.3 Administration Period

After completing the 2-week Run-in Period, subjects report to the clinical site for Day 1 procedures. During every study visit, subjects meet with a study dietician to review the subject's nutrition and activity patterns and determine if any deviations from their baseline patterns have occurred. The primary purpose of this dietary and physical activity review is to ensure that subjects are not vastly deviating from their usual patterns during the study, including but not limited to: initiating a new diet, introducing new meal items, stopping meals they were previously on, changing their exercise routine if they were routinely already on one, or not initiating a new exercise regimen (other than the protocol-required walking/physical activity). If deviations are occurring, subjects should be reminded of their baseline patterns, and encouraged to incorporate standard-of-care nutrition and activity recommendations to the best of their abilities.

The protocol-required 30 minutes of walking/physical activity at their usual pace or effort (in either three 10-minute walks/activity periods, two 15-minute walks/activity periods or one 30-minute walk/activity period), at least 3 days per week (i.e., at least 90 minutes per week), is reinforced at each study visit.

Study Food Product Information

### 4.1 Study Food Product Composition and Formulation Overview

CS-EMM-001 and placebo are packaged in individual stick packs. CS-EMM-001 is a mixture of selected amino acids formulated as dry powder. The composition of CS-EMM-001 with the total amount of amino acids contained in each stick pack is presented in Table 12 below. Placebo product is excipient and color-matched to the 53.9 g/day amount of CS-EMM-001.

TABLE 12
Amino Acid Composition of CS-EMM-001

| Amino Acid Component | Amount |
|---|---|
| L-Leucine | 0.8889 g |
| L-Isoleucine | 0.4444 g |
| L-Valine | 0.8889 g |
| L-Lysine Acetate | 0.4703 g |
| L-Histidine | 0.3333 g |
| L-Threonine | 0.3333 g |
| L-Ornithine Aspartate | 1.6667 g |

### 4.2 Study Food Product Administration

During the 12-week Administration Period, subjects consume their assigned study food product as instructed, which is administered orally by mixing the study food product in 8 oz. (˜240 mL) of water and consuming immediately after mixing. Daily administration of the study food product occurs three times per day with meals (±10 mins) every day for 12 consecutive weeks.

Table 13 below indicates the number of stick packs that are administered based on randomized group assignment. Subjects are instructed on how to prepare the study product and the number of stick packs that must be consumed at each meal. Administration diaries are reviewed with study subjects at each visit to ensure compliance with study product administration.

TABLE 13
Study Food Product Administration

| | Number of stick | | |
|---|---|---|---|
| | packs with | Number of stick | Number of stick |
| Administration | breakfast | packs with lunch | packs with dinner |
| Group | (±10 mins) | (±10 mins) | (±10 mins) |
| CS-EMM-001 | 2 | 2 | 2 |
| 29.4 g/day | | | |
| CS-EMM-001 | 4 | 3 | 4 |
| 53.9 g/day | | | |
| Placebo | 1 | 1 | 1 |

### 4.3 Study Blinding

This is a single-blind, placebo-controlled study. Subjects are blinded as to assignment to receive CS-EMM-001 or placebo. Site personnel dispensing the product to subjects for administration and the Sponsor are unblinded to product group assignment.

5 Study Assessments and Procedures

### 5.1 Physical and Neurological Examination

The Screening and Day 1/Baseline physical examination (PE) consists of an assessment of general appearance, skin, thorax/lungs, abdomen, lymph nodes, head, ears, eyes, nose throat, neck, and cardiovascular, musculoskeletal, and neurological systems. Subsequent PE examinations are abbreviated and consist of general appearance, skin, thorax/lungs, cardiovascular, abdomen, and neurological systems. The neurological examination consists of balance, gait, mental status, motor, and limited sensory assessments. The neurological examination is performed to identify any sign or symptom which could be directly or indirectly indicative of hepatic encephalopathy.

Physical and neurological examination findings are documented in the subject's source documents. Any new PE or neurological finding that represents a new abnormal finding or a worsening from Baseline condition will be recorded as an AE.

### 5.2 Vital Signs, Height and Weight, and Waist Circumference

Vital sign measurements include sitting systolic and diastolic blood pressure, heart rate, body temperature, and body weight and are measured at study visits. Height is measured at Screening and Day 1. Respiration rate is measured at the Baseline/Day 1, Week 6, Week 12 and Week 16 visits. BMI is calculated at every visit. Waist circumference is measured at every visit.

### 5.3 Mid-Upper Arm Muscle Circumference Measurements

The mid arm muscle circumference (MAMC) is a measure of muscle mass. It is calculated by taking a measurement of the total mid arm circumference (MAC) and deducting an amount for the layer of adipose tissue based on the triceps skin-fold (TSF). Measurement units for MAMC and MAC are in centimeters (cm) and TSF is in millimeters (mm). TSF is a measure of adiposity that is measured at the back of both arms, midway between the acromial process of the scapula and the olecranon process of the ulna, exactly at the same place where the mid arm muscle circumference is measured as indicated above.

Each time that the mid-arm circumference and triceps skinfold are measured, the MAMC will be calculated for each arm using the formula:

MAMC(cm)=MAC(cm)−[0.314×TSF(mm)]

### 5.4 Magnetic Resonance Imaging for Muscle Fat Infiltration

Subjects undergo an MRI examination up to 4 times during the study. MRI scans at Day 1, Week 6 and Week 12 are mandatory, and the MRI scan at Week 16 is optional.

Study MRI assessments include:
Total thigh muscle volume
The cross-sectional area of each thigh
The cross-sectional area of paraspinal and abdominal muscles at the level of the third lumbar vertebra (L3)
Muscle fat fraction within each thigh
Muscle fat fraction within all muscles at L3
Body composition profile, including visceral fat, subcutaneous fat, liver fat, muscle fat, total abdominal fat index, and lean muscle tissue.
   Imaging procedures follow established protocols.

While the invention has been particularly shown and described with reference to a preferred embodiment and various alternate embodiments, it will be understood by persons skilled in the relevant art that various changes in form and details can be made therein without departing from the spirit and scope of the invention.

All references, issued patents, and patent applications cited within the body of the instant specification are hereby incorporated by reference in their entirety, for all purposes.

What is claimed is:

1. A method for reducing fat infiltration in muscle comprising administering to a subject at risk of fat infiltration in muscle a composition comprising:
i) a histidine amino acid entity;
ii) a lysine amino acid entity; and
iii) a threonine amino acid entity;
wherein the composition does not include a physiologically effective amount of an amino acid entity selected from one, two, three, four, five, or all of a leucine amino acid entity, an isoleucine amino acid entity, a valine amino acid entity, a glutamine amino acid entity, an arginine amino acid entity, and a N-acetylcysteine amino acid entity (e.g., if leucine, isoleucine, valine, glutamine, arginine, or N-acetylcysteine are present, each is present at less than: 1 wt. %, 0.5 wt. %, 0.1 wt. %, 0.05 wt. %, 0.01 wt. %, 0.001 wt. %, or less, e.g., of the total wt. of the composition (e.g., in dry form)), with the proviso that the composition is not a composition of all essential amino acids (EAAs).

2. A method for reducing fat infiltration in muscle comprising administering to a subject at risk of fat infiltration in muscle a composition comprising:
a) a Branched Chain Amino Acid (BCAA) entity chosen from a leucine amino acid entity, an isoleucine amino acid entity, a valine amino acid entity, or a combination of two or three BCAA entities;
b) a Urea Cycle Amino Acid (UCAA) entity chosen from an ornithine amino acid entity chosen from L-ornithine, ornithine α-ketoglutarate, ornithine HCl, citrulline, or a combination thereof; an aspartate amino acid entity; or a combination of two UCAA entities; and
c) an essential amino acid (EAA) entity chosen from a histidine amino acid entity, a lysine amino acid entity, or a threonine amino acid entity or a combination of two or three EAA entities;
wherein:
i) at least one amino acid entity of (a)-(c) is not provided as a peptide of more than 20 amino acid residues in length;
ii) the total weight (wt.) % of (a)-(c) is greater than the total wt. % of non-amino acid entity protein components or other amino acid entity components in the composition on a dry weight basis; and
iii) two or more (e.g., all) of phenylalanine, tyrosine, or glutamine is absent from the composition, or if present, is present at less than 1 wt. % of the total wt. of the composition on a dry weight basis.

3. The method of claim 2, wherein the composition comprises:
a) the leucine amino acid entity is chosen from:
i) L-leucine or a salt thereof,
ii) a dipeptide or salt thereof, or tripeptide or salt thereof, comprising L-leucine, or
iii) β-hydroxy-β-methylbutyrate (HMB) or a salt thereof;
b) one or both of:
i) the ornithine amino acid entity is L-ornithine or a salt thereof or a dipeptide or salt thereof, or tripeptide or salt thereof, comprising L-ornithine; or
ii) the aspartate amino acid entity is L-aspartate or a salt thereof or a dipeptide or salt thereof, or tripeptide or salt thereof, comprising L-aspartate; and
c) the EAA entity is chosen from:
i) L-histidine or a salt thereof,
ii) a dipeptide or salt thereof, or tripeptide or salt thereof, comprising L-histidine,
iii) L-lysine or a salt thereof,
iv) a dipeptide or salt thereof, or tripeptide or salt thereof, comprising L-lysine,
v) L-threonine or a salt thereof, or
vi) a dipeptide or salt thereof, or tripeptide or salt thereof, comprising L-threonine.

4. The method of claim 1 or 2, wherein the subject suffers from cirrhosis of the liver.

5. The method of claim 1, wherein the composition further comprises a phenylalanine amino acid entity.

6. The method of claim 1 or 2, wherein the subject does not have elevated plasma levels of phenylalanine relative to a normal range of plasma phenylalanine from the Human Metabolomics DataBase (HMDB).

7. The method of claim 6, wherein the subject suffers from a condition other than cirrhosis of the liver.

8. The method of claim 1 or 2, further comprising determining a level of fat infiltration in thigh muscle.

9. The method of claim 1, wherein the composition does not include any of an amino acid selected from leucine, isoleucine, valine, glutamine, arginine, and N-acetylcysteine.

10. The method of claim 1, wherein the composition does not include a physiologically effective amount of each of leucine, isoleucine, valine, glutamine, arginine, and N-acetylcysteine.

11. The method of claim 10, wherein the composition does not include any of each of leucine, isoleucine, valine, glutamine, arginine, and N-acetylcysteine.

12. The method of claim 1, 5, 9, 10, or 11 wherein the composition reduces fat infiltration in myocytes cultured in vitro, e.g., in primary human differentiated myoblasts in medium designed to replicate a sarcopenic plasma profile and tested for lipid accumulation, e.g., as described in Example 2.

13. The method of claim 1 or 2, wherein administering the composition to the subject at risk of fat infiltration in muscle improves a muscle function of sequestering glucose.

14. The method of claim 13, wherein the subject at risk for fat infiltration in muscle has diabetes or metabolic disease.

15. The method of claim 1 or 2, wherein the subject at risk for fat infiltration in muscle suffers from cirrhosis with sarcopenia.

16. The method of claim 1 or 2, wherein the subject at risk for fat infiltration in muscle suffers from cirrhosis without sarcopenia.

17. The method of claim 1 or 2, wherein the subject at risk for fat infiltration in muscle has end-stage liver disease (ESLD).

18. The method of claim 17 wherein the ESLD includes one or more of hepatic encephalopathy, variceal bleeding, portal hypertension, ascites, infection risk, sepsis, all-cause hospitalization, and all-cause and liver-related mortality.

19. The method of claim 1 or 2, wherein the degree of fat infiltration in muscle is determined by magnetic resonance imaging (MRI).

20. The method of claim 1, wherein at least one of methionine (M), tryptophan (W), or cysteine (C) is absent, or if present, is present at less than a physiologically effective amount.

21. The method of claim 1, wherein the total wt. % of (i)-(iii) is greater than the total wt. % of any other single amino acid entity in the composition.

22. The method of any of the preceeding claims wherein the composition is a pharmaceutical composition and further comprises a pharmaceutically acceptable excipient.

23. The method of claim 1 or 2, which comprises evaluating fat infiltration in muscle in the subject to evaluate effectiveness of administration of the composition in treating the disease or disorder, decreased muscle function, or fracture or other trauma.

24. A method for determining whether a composition comprising a histidine amino acid entity, a lysine amino acid entity, and a threonine amino acid entity is effective in treating a disease or disorder associated with muscle function comprising determining whether there is a reduction in fat infiltration in muscle cells exposed to the composition.

25. The method of claim 24, wherein fat infiltration is in muscle tissue affected by the disease or disorder associated with muscle function.

26. The method of claim 24, wherein determining whether there is a reduction of fat infiltration in muscle cells exposed to the composition comprises evaluating the ability of the composition to reduce fat accumulation myocytes cultured in vitro, e.g., in primary human differentiated myoblasts in medium designed to replicate a sarcopenic plasma profile and tested for lipid accumulation, e.g., as described in Example 2.

27. The method of claim 24, wherein determining whether there is a reduction of fat infiltration in muscle cells exposed to the composition comprises administering the composition to the subject determining whether there is a reduction in fat infiltration in muscle cells in the subject.

28. The method of claim 27, wherein the determining step is determined by magnetic resonance imaging (MRI).

29. A composition comprising:
i) a histidine amino acid entity;
ii) a lysine amino acid entity; and
iii) a threonine amino acid entity;
wherein the composition does not include a physiologically effective amount of an amino acid selected from one, two, three, four, five, or all of a leucine amino acid entity, an isoleucine amino acid entity, a valine amino acid entity, a glutamine amino acid entity, an arginine amino acid entity, and a N-acetylcysteine amino acid entity (e.g., if leucine, isoleucine, valine, glutamine, arginine, or N-acetylcysteine are present, each is present at less than: 1 wt. %, 0.5 wt. %, 0.1 wt. %, 0.05 wt. %, 0.01 wt. %, 0.001 wt. %, or less, e.g., of the total wt. of the composition (e.g., in dry form)), with the proviso that the composition is not a composition of all essential amino acids (EAAs).

30. The composition of claim 29, wherein the composition further comprises a phenylalanine amino acid entity.

31. The composition of claim 29, wherein the composition does not include any of an amino acid selected from leucine, isoleucine, valine, glutamine, arginine, and N-acetylcysteine.

32. The composition of claim 29, wherein the composition does not include a physiologically effective amount of each of leucine, isoleucine, valine, glutamine, arginine, and N-acetylcysteine.

33. The composition of claim 29, wherein the composition does not include any of each of leucine, isoleucine, valine, glutamine, arginine, and N-acetylcysteine.

34. The composition of any one of claims 29-33, wherein the composition reduces fat infiltration, e.g., in primary human differentiated myoblasts in medium designed to replicate a sarcopenic plasma profile and tested for lipid accumulation.

35. The composition of claim 29, wherein at least one of methionine (M), tryptophan (W), or cysteine (C) is absent, or if present, is present at less than a physiologically effective amount.

36. The composition of claim 29, wherein the total wt. % of (i)-(iii) is greater than the total wt. % of any other single amino acid entity in the composition.

37. The composition of any of claims 29-36, wherein the composition is a pharmaceutical composition and further comprises a pharmaceutically acceptable excipient.

38. The composition of any of claims 29-36, wherein the composition is formulated as a dietary supplement (e.g., a nutritional supplement, a dietary formulation, a functional food, a medical food, a food, or a beverage).
